(19) United States
(12) Patent Application Publication
(10) Pub. No.: US 2005/0177183 A1
(43) Pub. Date: Aug. 11, 2005

(54) Guide-wire steered variable incision width safety scalpel

(76) Inventors: Gale H. Thorne, Bountiful, UT (US); Michael Wallace Howlett, Salt Lake City, UT (US); James Victor Mercer, West Jordan, UT (US)

Correspondence Address: Gale H. Thorne, 1056 Millcrest Circle, Bountiful, UT, 84010, US

(21) Appl. No.: 10/774,929

(22) Filed: Feb. 9, 2004

(51) Int. Cl. A61B 17/32; A61B 17/14

(52) U.S. Cl. 606/167; 606/181

(57) ABSTRACT

A variety of embodiments of a variable incision width guide-wire steered scalpel are disclosed. Each embodiment employs a split blade which is proximally hinged, thereby facilitating making a variable width incision of a predetermined, substantially constant depth. Methods of preselecting an incision width are disclosed. Also, a plurality of embodiments of such a scalpel which may be used to provide an incision of preselected depth, as well, are disclosed. Notably, once a depth has been predetermined, that depth is kept substantially constant over the width of a resulting incision. While a wire may be threaded through the scalpel as a guide, the scalpel may be used for other incision applications without a guide-wire. The scalpel has safety features which generally follow the steps of piercing to make an incision of predetermined depth, transversely opening the split blade to widen the incision to a predetermined width, closing split blade and retracting the blade into a protective housing. In some embodiments, the retracting step precedes the closing step.

### FIELD OF INVENTION

This invention relates generally to percutaneous incision devices and more particularly to scalpels and lancets which are precisely steered to an incision site via a guide-wire.

### BACKGROUND

Scalpels and lancets are well known in the medical arts. Lancets are commonly used for making incisions by patients themselves for blood sampling for, as examples, glucose and cholesterol level testing. Such lancets are provided in low cost embodiments to be self administered and are often found in safety and single use formats. Such lancets are generally directed to a somewhat randomly selected site which is purposely widely distributed so wounds do not overlap.

However, there is a special need for a more sophisticated instrument when introducing catheters into the vascular system. There is a wide range of central vascular procedures currently employed such as central venous catheters (CVC), cardiac catheterization, dialysis catheterization, angiography, and various interventional radiology procedures. Such catheters may be introduced into such body sites as elbow/upper arm, chest, neck or groin. Target vessels my be either arteries or veins or other body tubular structures.

As an example, when introducing a catheter using Seldinger or Micro-introducer techniques, a small diameter (e.g. 21 gauge) needle is used to puncture a vein or artery. A guide-wire (generally about 0.46 millimeters) is threaded to a target site in the vascular system. As is well known in the catheterization art, guide-wires facilitate traversing sometimes difficult arcuate turns in human vasculature.

Once the wire is so positioned, usually a larger catheter is threaded over the guide-wire to the target site. As the guide-wire introducer incision usually has a diameter which is consistent with the puncturing needle and/or the guide-wire, itself, orifice size at the incision site is often much smaller than the larger catheter or an associated catheter introducer used to facilitate catheter introduction. Such catheter introducers are generally larger than the associated catheter and are now commonly designed to be “peeled away” from the catheter after introduction.

Currently, it is common practice to “nick” tissue about an incision site to increase the entry orifice diameter to permit easier introduction of a catheter about an inserted guide-wire. As an example, a scalpel (e.g. with a number 11 blade) is commonly employed, although some clinicians my forego nicking and depend upon tissue resiliency by forcing the sheath introducer through the skin in a corkscrewing fashion. A few clinicians use other methods for increasing orifice size, such as by nicking with an 18 gauge needle.

All such practices require great skill to nick precisely enough to not inadvertently over-extend incision size and yet achieve an orifice sized for desired ease of catheter introduction. Current techniques for nicking vary widely. Some technicians nick vertically while others nick horizontally. It is most common to nick below a guide-wire, but some may nick over the wire or even make multiple nicks at right angles.

Depth of nicking is also of concern. As examples, a superficial vessel insertion may require a shallow nick (approximately 3 millimeters) while a femoral artery introduction may require a deeper (approximately 10 millimeter) nick. Problems including blade sharpness and skin toughness combine to cause nicking inaccuracies and errors.

Incision width (entry orifice size) may also vary (e.g. for 3 to 18 french catheter sizes) and for introducers through which the catheters are inserted. For these reasons, it is highly desirable to provide a single incision-making instrument which may be used to provide accurate and precise variably selected depth and width nicks at a site accurately determined by an inserted guide-wire.

It should be noted that an algebraic relationship exists between incision width and diameter of a catheter to be inserted. As an example, a size 10 french catheter is nominally 0.131 inches (3.33 millimeters) in diameter(d). Therefore, for a size 10 french catheter an incision width of 0.206 inches (5.23 millimeters) should be provided. Relationship to incision width(w) to catheter diameter(d) may be calculated by the following equation:
w=π/2
where π=3.142.

In rudimentary form, over guide-wire use of a scalpel is taught in a U.S. Pat. No. 5,843,108, titled OVER THE WIRE SCALPEL, issued Dec. 1, 1998 to Samuels (Samuels), in a U.S. Pat. No. 4,633,860, titled CANAL FORMING DEVICE FOR PERCUTANEOUS NEPHROSCOPY, issued Jan. 6, 1987 to Korth et al. (Korth) and in another U.S. Pat. No. 4,955,890, titled SURGICAL SKIN INCISION DEVICE, PERCUTANEOUS INFECTION CONTROL KIT AND METHODS OF USE, issued Sep. 11, 1990 to Yamamoto, et al.(Yamamoto).

Samuels discloses a scalpel having a triangular shaped unitary blade having a pair of cutting edges which meet to define a tip. It may be noted that such a blade cannot produce a variable width incision at a predetermined, constant depth.

Korth discloses one or more scalpel blades securely affixed to and radially extending outward from a tubular member which can be slideably displaced over a guide wire. Incisions resulting from use of the one or more scalpel blades of Korth are of fixed width.

Yamamoto teaches a pair of blades which form a cutter. Each blade of the pair of blades of Yamamoto lies on a side of a groove through which a guide-wire is threaded. Similar to the blade of Samuels, the combination of the pair of blades of Yamamoto cannot produce a variable width incision at a predetermined, constant depth, much less controllably vary both width and depth of an incision.

Varying depth of an incision between a more shallow and a deeper cut is disclosed in U.S. Pat. No. 4,759,363 titled SCALPEL WITH REMOVABLE DEPTH GUARD, issued Jul. 26, 1988 to Jensen (Jensen). Jensen teaches the use of a single removable guard which is used to transform the scalpel for cutting to a lesser, second depth of cut when the guard is affixed to the scalpel.

### BRIEF SUMMARY AND OBJECTS OF THE INVENTION

In brief summary, this novel invention alleviates all of the known problems related to nicking at a guide-wire (catheter) introduction site. It is well known in medical practice that overextending width and/or depth of an incision may result in serious tissue, vein or even arterial damage with subsequent pain and bleeding. Further, an incision width which is significantly wider than diameter of an inserted catheter, increases risk of infection.

A scalpel instrument made according to the instant invention provides for accurately and precisely enlarging a wound to a predetermined incision width at a guide-wire introduction site preparatory to insertion of a catheter or catheter introducer. Notably, such a scalpel instrument may also be used to provide a method for a quick and accurate depth/width controlled incision without involving a guide-wire. An example of such use is incising skin prior to invasive scope introduction.

A scalpel instrument made according to the invention comprises a protective housing for a scalpel blade and an actuator used to displace the blade. The scalpel blade is a proximally hinged, split blade having two distally disposed sharpened, pointed ends. It should be understood that, hereafter, the term “blade” referenced as a blade associated with the instant invention is defined to be a scalpel blade. Further, the instrument housing comprises a pathway through which a guide-wire is slidably disposed to provide a steering line for the scalpel and blade to an insertion site of the guide-wire.

The blade, housing and actuator interact to displace the blade through two distinct modes of action as the actuator is manually displaced to drive sharpened points of the blade from the housing. A first mode thrusts the blade distally outward from the housing and toward a guide-wire insertion site and extends the blade points to a preselected depth with a minimal incision width. Once the preselected depth is reached, the split blade is spread transversely to widen the incision to a predetermined width. An interface between the housing and blade is geometrically configured to maintain the resulting incision at a constant depth as the actuator continues to drive the blade apart to widen the incision.

Preferably, the guide-wire pathway is slanted relative to a plane of direction of blade displacement to provide coincidence between the guide-wire and the exposed sharpened points of the blade. For improved safety, a spring may be disposed between a housing connection and a part of the actuator structure to retract the blade when force upon the actuator to displace the scalpel blade is relieved. Also, in one embodiment, the blade points are maintained in an apart state while being retracted to minimize interaction between blade and guide-wire during blade retraction.

To meet cost objectives, several parts of a scalpel made according to the invention may be made in a single unitary mold. As an example, a housing of two parts and an actuator may be made as a single molded part. The blade may be stamped, bent to shape and honed to produce necessary sharp points and edges by methods which are well known in blade manufacturing art.

A “nose” part of a scalpel made according to the instant invention may be variably displaced to select a predetermined depth of penetration of the scalpel blade. Note in this case, actuator displacement is constant for all selected depths of penetration. However, in the second mode of blade displacement, shortening actuator blade travel limits blade point separation and, therefore, incision width. For this reason precise stops are provided to abbreviate actuator displacement and, therefore, blade separation and resulting incision width. Such stops, permit a user to drive the actuator until the stop is reached with full knowledge the incision width will not exceed that desired stop setting.

Accordingly, it is a primary object to provide a scalpel which is steered by a guide-wire to an incision site.

It is an important object to provide a scalpel having a guide-wire pathway through the scalpel.

It is an important object to provide a scalpel having a scalpel blade with a sharpened point which is longitudinally displaceable relative to the guide-wire.

It is another important object to provide a pathway which cooperatively guides the scalpel along the guide-wire such that the point of the blade of the scalpel upon exiting the scalpel coincides with the guide-wire.

It is a fundamental object to provide a scalpel blade which is longitudinally split to permit spreading of the scalpel to vary width of a resulting incision.

It is another fundamental object to provide a hinged scalpel blade having two articulating elongated parts each having a sharpened point and transverse knife edge.

It is yet another fundamental object to provide an actuator which, in cooperation with the housing, drives the scalpel blade along a plane in a first mode which determines incision depth with a minimal incision width and then in a second mode which, in the same plane, drives the separable elongated parts apart to further widen the incision to a preselected width.

It is yet another fundamental object to provide the scalpel blade parts with a common hinge whereby the parts are hinged together to be articulated and, thereby, angularly separated about the hinge.

It is another basic object to provide a geometric interface between the housing and scalpel blade which maintains a substantially constant incision depth as the width of the incision is varied.

It is an object to provide a housing comprising top and bottom parts which are injection molded.

It is an object to provide an actuator which interfaces with the housing and blade to permit manual displacement of the blade relative to the housing.

It is an object to provide an actuator which comprises a plurality of hinged parts whereby a mechanical advantage is derived whereby manual distance of displacement to displace the blade is less than associated distance of displacement of the blade.

It is an object to provide an actuator having a plurality of hinged parts which is molded as a single unitary part with hinges being living hinges.

It is an object to provide an actuator and at least one housing part molded as a unitary part with a living hinge interconnection between housing part and actuator.

It is an object to provide a housing having a top part and a bottom part which are interconnected by a living hinge and finally assembled by closure likened to closure of a clam shell.

It is an object to provide a displaceable “nose” affixed to the housing of the scalpel whereby the nose is displaced a predetermined amount relative to the blade to vary incision depth to a preselected amount.

It is a very important object to provide a selectable stop for the actuator to limit scalpel blade separation, thereby limiting incision width to a predetermined length.

It is an object to provide at least one latch which retains the scalpel blade in a housed disposition until the actuator is advertently displaced.

It is an object to provide a scalpel embodiment which is open to receive a guide-wire and then closed to capture the guide-wire in the pathway prior to use in a medical procedure.

It is an object to provide another scalpel embodiment which is closed prior to communicating with a guide-wire, the guide-wire being threaded through a pathway, disposed in the housing, prior to use in a medical procedure.

It is a very important object to provide a safety scalpel comprising a memory element in which energy is stored upon outward displacement of the blade from the housing (forward displacement of the actuator) an which releases the energy to return the blade upon relief of force being placed upon the actuator.

It is an object to provide a safety scalpel which maintains retracting scalpel blades separated apart state until potential contact with a guide-wire is obviated.

These and other objects and features of the present invention will be apparent from the detailed description taken with reference to accompanying drawings.

### BRIEF DESCRIPTION OF THE DRAWINGS

FIG. 1 is a perspective of a first embodiment of a closed guide-wire steered variable incision width scalpel made according to the instant invention.

FIG. 1A is a perspective of the scalpel seen in FIG. 1 with a portion of a scalpel blade seen exposed from a distal end of the scalpel.

FIG. 1B is a perspective of the scalpel seen in FIGS. 1 and 2 with blade portions of the scalpel separated for the purpose of widening an incision after the scalpel blade is introduced into a catheter entry site.

FIG. 2 is a top elevation of a second embodiment of a closed guide-wire steered variable incision width scalpel which is similar in form and function to the scalpel seen in FIGS. 1, 1A and 1B, but having a different set of indicia seen thereon.

FIG. 2A is a top elevation of the second embodiment of the scalpel seen in FIG. 2 with an actuator displaced to expose a split scalpel blade contained therein.

FIG. 2B is a top elevation of the second embodiment of the scalpel seen in FIG. 2A with the actuator further displaced to separate parts of the split blade.

FIG. 3 is a segment of a cross section taken along lines 3-3 of FIG. 2 at a plane disposed at the top level of the split scalpel blade seen in FIG. 2A.

FIG. 3A is the segment of the cross section seen in FIG. 3 with the blade forced distally by displacement of a pair of studs, disposed in slots of the blade to expose tips of the scalpel blade (as are also seen in FIG. 2B).

FIG. 3B is the segment of the cross section seen in FIG. 3A with the pair of studs forced inwardly through the plane of the blade.

FIG. 3C is the segment of the cross section seen in FIG. 3B with the pair of studs further distally displaced to produce a gap between tips of the split scalpel blade.

FIG. 3D is the segment of the cross section seen in FIG. 3C with the pair of studs still further distally displaced to widen the gap between the tips of the split scalpel blade.

FIG. 3E is the segment of the cross section seen in FIG. 3D with the pair of studs still further distally displaced to additionally widen the gap between the tips of the split scalpel blade.

FIG. 4 is a perspective of an actuator whereupon two studs, which are disposed within slots of a split scalpel blade as seen in FIGS. 3 and 3A-E, are seen.

FIG. 5 is a section taken along lines 5-5 of FIG. 4 of an actuator affixed to a scalpel; the actuator is seen having studs disposed through a split scalpel blade and into grooves in the base of a housing.

FIG. 5A is a section similar to the section of FIG. 5, with actuator studs displaced through the blade into a deepened groove in the housing.

FIG. 6 is a perspective of an open scalpel housing with a blade disposed therein and associated studs affixed in slots in the blade.

FIG. 7 is a top elevation of a split scalpel blade, prior to being bent and otherwise prepared for use.

FIG. 7A is a side elevation of the scalpel blade seen in FIG. 7, bent to provide a vertical proximal hinge and channel for a guide-wire.

FIG. 7B is a top elevation of the blade seen in FIG. 7A with a distal end honed to provide a sharpened entry point.

FIG. 8 is a perspective of the open scalpel housing seen in FIG. 6 with a guide-wire medially disposed across the blade within the guide-wire channel.

FIG. 9 is a perspective of the open scalpel housing seen in FIG. 6 with a straw (shown by dashed lines medially displaced across the blade within the guide channel) which provides a pathway for steering a guide-wire through a scalpel housing after closure.

FIG. 9A is a perspective of the straw seen as dashed lines in FIG. 9.

FIG. 10 is a perspective of an open housing and associated actuator with a spring affixed between the housing and actuator to provide retractive force to return a scalpel blade to the housing after use.

FIG. 11 is a top elevation of a third embodiment of a Split blade scalpel with facility (holes) for a physically changeable actuator displacement limiter being disposed thereupon.

FIG. 11A is a top elevation of the scalpel seen in FIG. 11 with an actuator displacement limiter disposed to limit blade separation to a 3 french scale width.

FIG. 11B is a top elevation of the scalpel seen in FIG. 11 with a actuator displacement limiter disposed to limit blade separation to a 4 french scale width.

FIG. 11C is a top elevation of the scalpel seen in FIG. 11 with a actuator displacement limiter disposed to limit blade separation to a 17 french scale width.

FIG. 12 is a segment of a cross section taken along lines 12-12 of FIG. 11 at a plane disposed at the top level of the split scalpel blade seen in FIGS. 11A-C.

FIG. 12A is the segment of the cross section seen in FIG. 12 with the blade forced distally by displacement of a pair of studs, disposed in slots of the blade to displace the blade and thereby expose tips of the scalpel blade.

FIG. 12B is the segment of the cross section seen in FIG. 12A with the pair of studs forced distally to separate tips of the split scalpel blade.

FIG. 12C is the segment of the cross section seen in FIG. 12B with the pair of studs further distally displaced to produce a widened gap between tips of the split scalpel blade.

FIG. 12D is the segment of the cross section seen in FIG. 12C with the pair of studs still further distally displaced to additionally widen the gap between the tips of the split scalpel blade.

FIG. 12E is the segment of the cross section seen in FIG. 12D with the pair of studs still further distally displaced to yet additionally widen the gap between the tips of the split scalpel blade.

FIG. 13 is a perspective of an actuator associated with the scalpel of FIGS. 11 and 12.

FIG. 14 is a perspective of a blade having a lengthened tip section and providing a visual example of one method of controlling depth of insertion of tips of a blade by varying displacement of a nose section, indicated by dashed lines.

FIG. 14A is a split scalpel blade which is similar, but simpler in construction than the blade seen in FIG. 7B.

FIG. 15 is a perspective of another embodiment of a closed guide-wire steered variable incision width scalpel, made according to the instant invention, wherein an actuator has a relatively long displacement relative to the actuator displacement of the scalpel of FIG. 1 to deepen an incision.

FIG. 16 is a perspective of still another embodiment of a closed guide-wire steered variable incision width and depth scalpel made according to the instant invention.

FIG. 16A is a sectional portion removed along lines 16A-16A of FIG. 16.

FIG. 16B is a perspective of the scalpel seen in FIG. 16 with a guide-wire inserted through the scalpel and actuator displaced to expose tips of the split scalpel blade.

FIG. 16C is a perspective of the scalpel seen in FIG. 16B with the actuator further displaced to widen a gap between the split scalpel blades.

FIG. 17 is a perspective of a split scalpel blade used in the scalpel embodiment seen in FIGS. 16, 16B and 16C.

FIG. 18 is a section taken along lines 18-18 of FIG. 16.

FIG. 19 is a section taken along lines 19-19 of FIG. 16.

FIG. 20 is a section taken along lines 20-20 of FIG. 16.

FIG. 21 is a perspective of a top view of a displace able nose part of the scalpel seen in FIG. 16.

FIG. 21A is a perspective of a bottom view of the displace able nose part seen in FIG. 21.

FIG. 22 is a top elevation of a unitary molded section which includes a bottom portion of a housing and an actuator for the scalpel seen in FIG. 16; note that a retracted blade is seen disposed within the housing.

FIG. 22A is a top elevation of a unitary molded section which includes a bottom portion of a housing and an actuator for the scalpel seen in FIG. 16; note that an extended blade is seen disposed within the housing.

FIG. 22B is a top elevation of a unitary molded section which includes a bottom portion of a housing and an actuator for the scalpel seen in FIG. 16; note that an extended and hingedly separated blade is seen disposed within the housing.

FIG. 22C is a bottom elevation of a unitary molded section which includes a bottom portion of a housing and actuator for the scalpel seen in FIG. 16.

FIG. 23 is a perspective of a top part of the scalpel seen in FIG. 16.

FIG. 24 is a perspective of a blade, similar to the blade seen in FIG. 17, but comprising features used to permit blade sharpened ends to remain apart as the blade is retracted into a housing.

FIG. 25 is a top elevation of a bottom portion of a scalpel housing made according to the invention with the blade seen in FIG. 24 disposed in a before-used state.

FIG. 25A is a top elevation of the bottom portion and blade of FIG. 25 with the blade displaced outward from the housing and sharpened ends of the blade sharpened ends articulated apart.

FIG. 25B is a top elevation of the bottom portion and blade of FIGS. 25 and 25A with the blade retracted while the sharpened ends remain articulated apart.

FIG. 26 is a medial section similar to the section of FIG. 19, but of a scalpel having a blade and housing as seen in FIG. 25, whereby initial disposition of the scalpel blade is seen.

FIG. 26A is a section similar to the section of FIG. 20, but of the scalpel having parts seen in FIGS. 25A and 25B with wing portions of the blade seen in various stages of blade displacement.

### DETAILED DESCRIPTION OF THE ILLUSTRATED EMBODIMENTS

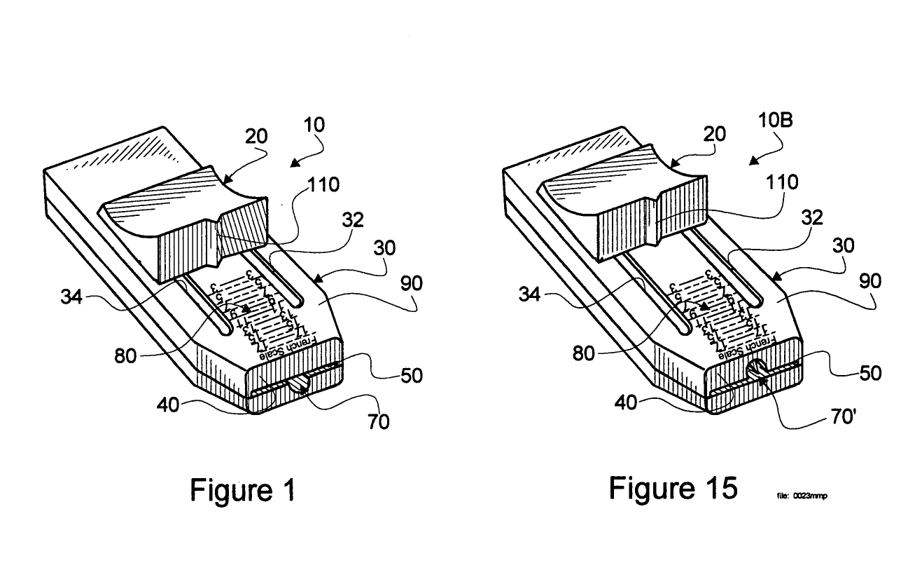

Unless otherwise specified, the term proximal is used to indicate a portion or segment of a referenced device normally facing or near a clinician or other person using the device. The term distal refers to a portion or segment of a referenced device which is generally away from the clinician or other person using the device. Reference is now made to the embodiments illustrated in FIGS. 1-26A wherein like numerals are used to designate like parts throughout. As a number of embodiments are disclosed herein, parts which are similar in form and function are given like numbers, but differentiated one from the others by a unique letter or a prime assigned to the part number. As an example, a part like, but functionally or formed somewhat differently than a part numbered 3 may be assigned the number 3A or the number 3′.

Adjustable Incision Width Scalpel

Reference is now made to FIGS. 1, 1A and 1B wherein a first embodiment of a guide-wire steered variable incision width scalpel, numbered 10, is seen. As seen in FIG. 1, scalpel 10 comprises a manually operated actuator 20 and a scalpel housing 30. Housing 30 comprises a pair of elongated slots, numbered 32 and 34, through which actuator 20 communicates with a scalpel blade (seen in FIGS. 1A and 1B). At its distal end 40, housing 30 comprises an elongated slot 50. Distal displacement of actuator 20, seen accomplished in FIG. 1A, projects a split scalpel blade 60 outwardly through slot 50.

Note that blade 60 comprises a pair of juxtaposed sharpened tips 62 and 64. As seen in FIG. 1B, further displacement of actuator 20 causes tips 62 and 64 to separate. Note that, in this manner, scalpel 10 operates in two modes. The first mode is projection of a closed split blade outward (from a scalpel which has been previously disposed at an incision site which may be a guide-wire entry site) to make an incision of minimal width and fixed, predetermined depth. Once the predetermined depth is attained, in the second mode, juxtaposed sharpened tips 62 and 64 of the split blade are forced apart to widen the resulting nick to a controlled, predetermined width.

Note an orifice 70 in close proximity to slot 50 is disposed to provide an entry for passage of a guide-wire (not shown in FIGS. 1, 1A or 1B). This close proximity of slot 50 to an entry orifice for a guide-wire permits the scalpel to be aligned and, thereby, be displaced closely to the guide-wire at the site of an entry incision where the guide-wire has previously been introduced. This permits the scalpel to be used to accurately enlarge an incision at a wound site to facilitate per cutaneous insertion of a catheter of larger diameter than the original wire insertion wire entry incision.

As it is currently common practice to use a scalpel to nick an incision about a guide-wire to enlarge access diameter at the guide-wire entry site, it is well understood by those having experience that such a practice requires significant training and experience to open the incision to a desired size without making the resulting wound too large. As indicated by indicia 80, affixed to the top side 90 of housing 30, limiting displacement of actuator 20 to a predetermined distance along top 90 consequently limits sideways displacement and resulting separation of blades 62 and 64 in a scalpel made in accordance with the instant invention.

One of the remarkable features which may be achieved by a scalpel made according to the invention is not only providing a cut which is of known depth relative to a per cutaneous entry site, but maintaining substantially the same depth of cut across the full width of the slice, independent of width of incision. Method and apparatus for achieving a constant depth of incision is disclosed in detail hereafter.

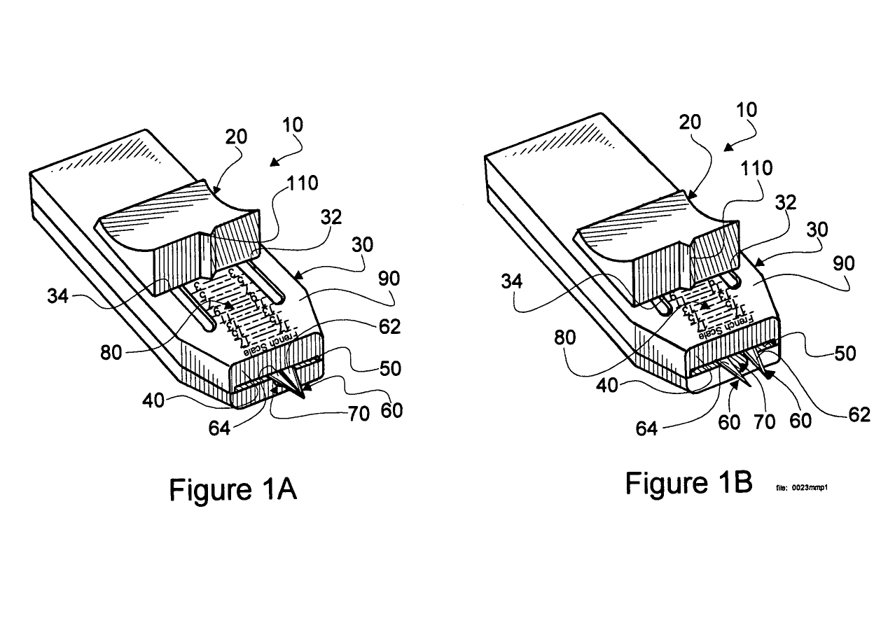

Reference is now made to FIGS. 2, 2A and 2B which provide a more complete disclosure of the two modes of scalpel operation. In FIG. 2, a scalpel 10A is seen to comprise a layout of indicia 80A which is different than the layout of indicia 80 seen in FIG. 1. Also, in FIGS. 2, 2A and 2B, actuator to blade communicating slots are hidden by an actuator 20A. In addition, in FIGS. 2, 2A and 2B, a guide-wire 100 is seen to be disposed through housing 30A.

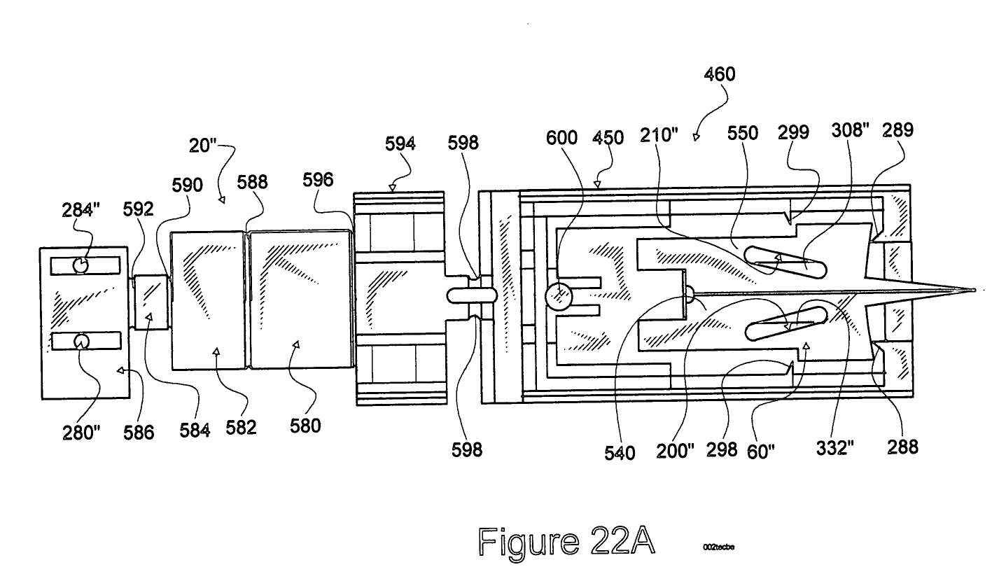

In use, scalpel 10A is displaced along guide-wire, generally numbered 100, until distal end 40A is disposed to reside against a skin interface at an incision site. Once so disposed, actuator 20A is manually displaced until a pointer 110 (associated with a most distal site on actuator 20A) indicates a minimal incision width (as seen in FIGS. 2 and 2A to be 3 french) has been reached. Note that, at minimal incision width, travel of actuator 20A is equal to travel distance of blade 60. Further travel of actuator 20A (or actuator 20) only separates blade tips 62 and 64 to produce a wider incision as seen in FIG. 20B. Note also that separated blade tips 62 and 64, in FIG. 2B, are disposed at the same depth of penetration as depth of penetration seen in FIG. 2A.

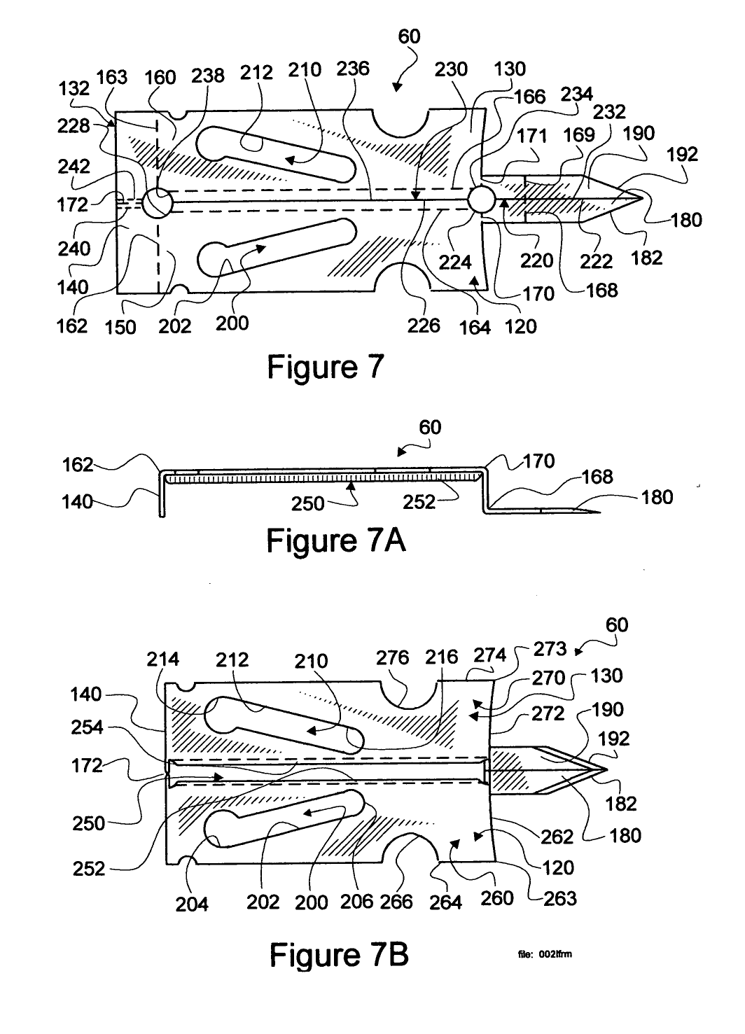

A blade 60 made in accordance with the instant invention for use in scalpels 10 and 10A is variously seen in FIGS. 7, 7A and 7B. As seen in FIG. 7, blade 60 may be formed by stamping from medical grade stainless steel or other material from which a sharpened scalpel blade may be made. Blade 60 is seen to be formed as two juxtaposed, elongated components 120 and 130 which are joined at a proximal end 132 by a hinge segment 140. Hinge segment 140, is proximally integrally joined to a planar portion, numbered 150 and 160, respectively, of each component 120 and 130. Note that top faces of portions 150 and 160 lie in a common plane and are juxtaposed about a medial plane which is orthogonal to the common plane. A number of bend delineations are seen as dashed lines, 162, 163, 164, 166, 168, 169, 170, 171 and 172. It is anticipated that stamping and bending may be performed simultaneously, although such is not necessary within the scope of the instant invention.

Further blade 60 comprises a series of features which are useful in providing a scalpel blade which may be used to penetrate an incision site to a constant depth and then to be transversely driven to widen the wound to a predetermined desired width. For this purpose, as an example, component 120 has a sharpened end 180 which may be honed along a transverse edge 182 (see FIG. 7B) to form a sharpened blade. Similarly, component 130 has a sharpened end 190 which may be honed along a transverse edge 192 (see FIG. 7B) to form a sharpened blade. Note that it may be possible to provide edges 182 and 192 in sharp enough condition for cutting by stamping alone, although such is not recommended.

As seen in FIG. 7B, proximally disposed from sharpened end 180, component 130 comprises an elongated slot 200. Slot 200 is bounded outwardly from the medial plane by an edge 202 which is angularly disposed relative to the medial plane such that a proximal end 204 of edge 202 is further from the medial plane than a distal edge 206. Further, at proximal end 204, slot 200 is enlarged for purposes which are disclosed in detail hereafter.

In like manner, proximally disposed from sharpened end 190, component 130 comprises an elongated slot 210. Slot 210 is bounded outwardly from the medial plane by an edge 212 which is angularly disposed relative to the medial plane such that a proximal end 214 of edge 212 is further from the medial plane than a distal edge 216. Further, at proximal end 214, slot 210 is enlarged for purposes which are also disclosed in detail hereafter.

Component 120 comprises a medially disposed, component 130 facing edge 220 (see FIG. 7) which is formed by a linear segment 222, an arcuate segment 224, proximal linear segment 226 and another edge segment 228. Likewise, component 130 comprises a medially disposed, component 120 facing edge 230 which is formed by a distal linear segment 232, an arcuate edge 234, proximal linear segment 236 and another arcuate segment 238. Segments 222, 226, 232 and 236 are component 120 to 130 interfacing elements disposed in both the common and medial planes when blade 60 is disposed as seen in FIGS. 7 and 7B. Note that arcuate segments 228 and 238 join in segment 140.

Edges 224 and 234 provide relief at bend lines 170 and 171 such that blade 60 may be formed as seen in FIG. 7A. (Only bend line 170 is seen in FIG. 7A.) In similar fashion, edges 228 and 238 provide relief at bend lines 162 and 163, respectively, such that blade 60 may be formed at a proximal end as seen in FIG. 7A. (Only bend line 162 is seen in FIG. 7A.) Note that bending hinge portion 140 orthogonal to the common plane forms a hingeable region about dashed line 172 and thereby permits components 120 and 130 to be articulated about hinge line 172. To make articulation more sure, it is preferred to thin a segment of hinge portion 140 medially (e.g. between dashed lines 240 and 242, see FIG. 7) during the stamping process.

Blade 60 is formed by bending components 120 and 130 at bend lines 170 and 171, respectively, to displace sharpened ends 180 and 190 away from the common plane and bent again along bend lines 168 and 169, respectively, to return sharpened ends 180 and 190 to a direction which is parallel to the common plane. As is disclosed hereafter, a guide-wire along which scalpel 10 may be steered can be disposed in a channel 250 formed by bending components 120 and 130 along lines 164 and 166, respectively. As seen in FIG. 7B (and in FIG. 7A) elongated channel portions 252 and 254 are downwardly bent to form channel 250 wherein a guide-wire may be placed to facilitate use of scalpel 10.

Reference is now made to FIG. 7B wherein a distal portion of component 120 is seen to comprise a wing 260. On a distal side, wing 260 is bounded by an edge 262 which extends outwardly from a more medial contact with sharpened end 180 to join, at corner 263, an outwardly disposed, straight edge 264 which is substantially parallel to the medial plane when scalpel 10 is disposed in a closed state, as seen in FIG. 7B. Proximally, edge 264 is joined to an arcuate, medially displaced edge 266.

Similarly, component 130 comprises a wing 270. Wing 270 is distally bounded by an edge 272 which extends outwardly from a more medial contact with sharpened end 190 to join, at corner 273, an outwardly disposed, straight edge 274 which is substantially parallel to the medial plane when scalpel 10 is disposed in the closed state of FIG. 7B. Proximally, edge 274 is joined to a medially displaced edge 276.

Distally disposed edges 262 and 272 provide interfaces for stops contained within housing 30 (or 30A) to limit distal travel of blade 60. Straight edges 264 and 274 interface with side stops disposed in housing 30 to maintain blade 60 in a closed state (as seen in FIG. 7B) until the maximum distal travel of blade 60 is achieved. At such a point, each edge, 266 and 276, interfaces with side stops to permit wings 260 and 270 and therefore sharpened ends 180 and 190 to be displaced transversely.

Careful attention to edges 262 and 272 reveals that each such edge has a curvature (i.e. is not a straight edge). Each edge 262 and 272 has a curvature which permits a change in distal displacement of blade 60 to compensate for blade angulation as incision width is changed. Thereby sharpened ends 180 and 190 may be maintained at a constant penetration depth independent of width of an incision made as components 120 and 130 are articulated outward away from the medial plane.

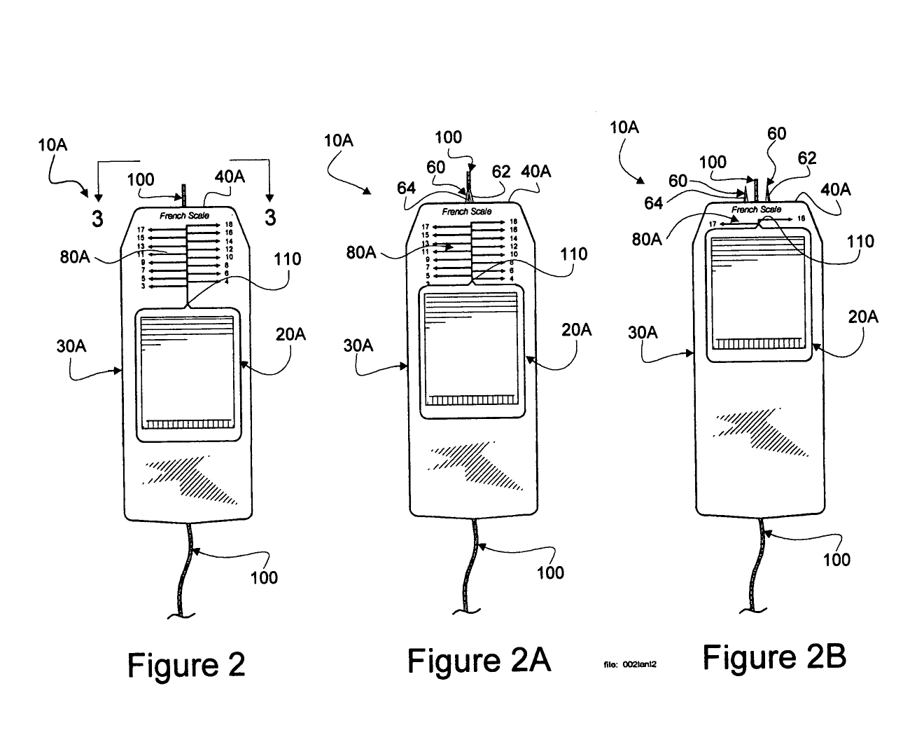

Reference is now made to FIGS. 3, 3A-3E, 4, 5 and 5A. Seen in FIGS. 3 and 3A-3E is a section of a portion of housing 30 made across the common plane. A number of details of scalpel 10 are not shown in FIGS. 3 and 3A-3E for simplicity of presentation of actuation of scalpel blade 60.

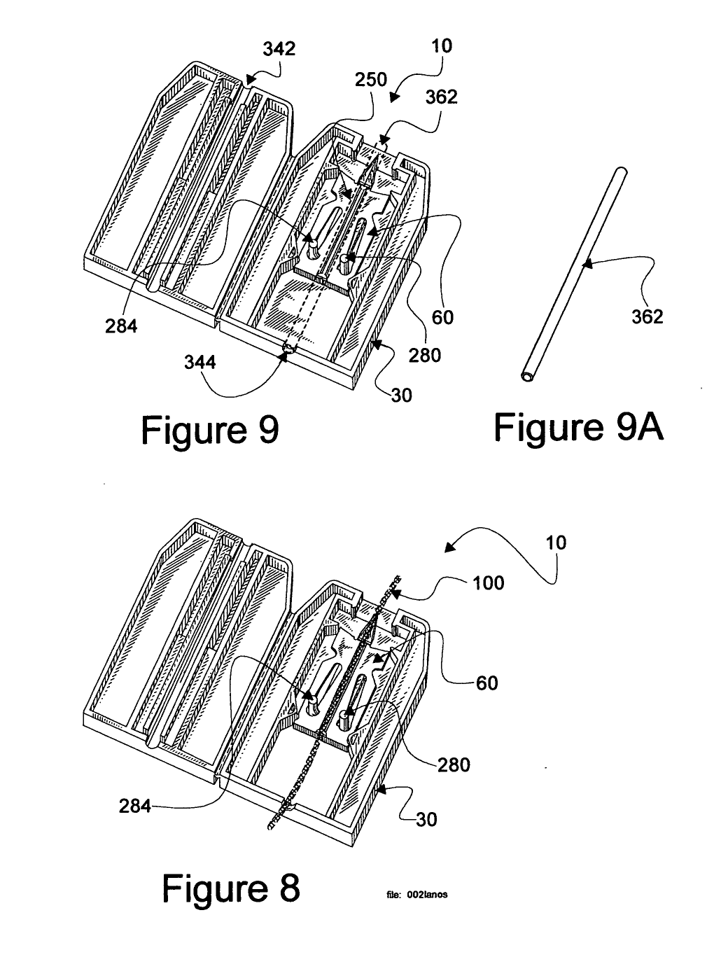

In FIG. 3, note a stud 280 disposed in an enlarged portion 282 of slot 200. Similarly, a stud 284 is disposed in an enlarged portion 286 of slot 210. Enlarged portions 282 and 286 serve the following two purposes. First enlargement permits penetration of studs 280 and 284, each having a retaining latch which is disclosed in detail hereafter. Secondly, each stud 280 and 284 has a variable diameter which is sharply increased at that section of the stud (numbered 290 and 292, respectively) which is further from the main body of the actuator and seen in FIG. 8, also disclosed in detail hereafter.

As seen in FIGS. 3A-3E, distal displacement of actuator 20, is in line with the medial plane and drives studs 280 and 284 along lines parallel to the medial plane. Note, in FIGS. 3 and 3A, cross sectional size of each section 290 and 292 of each respective stud 280 and 284 does not permit either stud from being displaced out of portions 282 and 286, respectively. For this reason, distal displacement of actuator 20 results in a like distal displacement of blade 60.

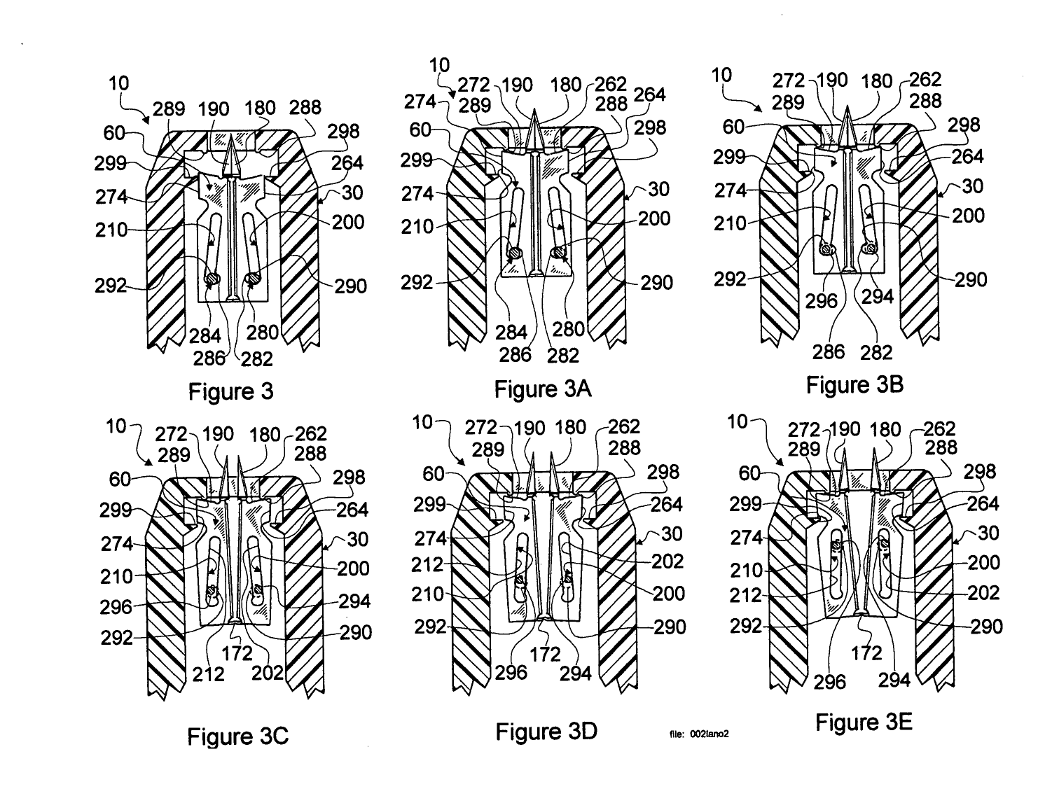

However, when each stud 280 and 284 is displaced (upwardly from the plane of the referenced figures) a smaller diameter portion (numbered 294 and 296 is revealed). See FIGS. 3B and 4. In FIGS. 3B-3E, the larger diameter sections 290 and 292, residing outside portions 282 and 286, respectively, are shown as dashed lines. As seen in FIGS. 3C-3E, continued distal displacement of actuator 20 drives the smaller segments 294 and 296 along edges 202 and 212, respectively, to displace sharpened ends 180 and 190 apart and, thereby, provide an incision widening action.

As in scalpel 10, incision width is determined by a visual measurement made by viewing position of pointer 110 (see FIGS. 1 and 2) relative to indicia (e.g. 80 and 80A). It is advisable to provide a controlling step between the two modes of scalpel operation. As disclosed supra, larger sections 290 and 292 of studs 280 and 284 should remain in portions 282 and 286 (see FIG. 3) until a deepest point of an incision is reached (see FIG. 3A). Note that, at the deepest point of an incision, blade 60 is restricted from further distal displacement by a pair of bulbous projections forming stops 288 and 289 which restrictively communicate with edges 262 and 272, respectively (see FIG. 3A). Note, also in FIGS. 3C-3E, that curvature of edges 262 and 272 maintain respective sharpened ends 180 and 190 at a constant incision depth as blade 60 is angularly displaced.

At that point, studs 280 and 284 should be adjusted relative to the common plane to displace sections 294 and 296 into portions 282 and 286 (see FIG. 3B). Note also in FIG. 3A that when blade 60 is fully displaced against stops 288 and 289, edges 264 and 274 are respectively displaced from contact with two side stops 298 and 299. So displaced relative to stops 298 and 299, blade 60 is freed for arcuate separation as seen in FIGS. 3C-3E.

As seen in FIG. 4, actuator 20 comprises a manual actuator button 300 having a top surface 302 which provides a manual digitary interface. Projecting outward from a bottom surface 304 of actuator 20 are studs 280 and 284. Each stud 280 and 284 has a thinned section 294 and 296, respectively, and an enlarged section 290 and 292 as disclosed supra. Further, each stud 280 and 284 has an inwardly disposed latch 306 (seen in FIG. 4 only upon stud 284).

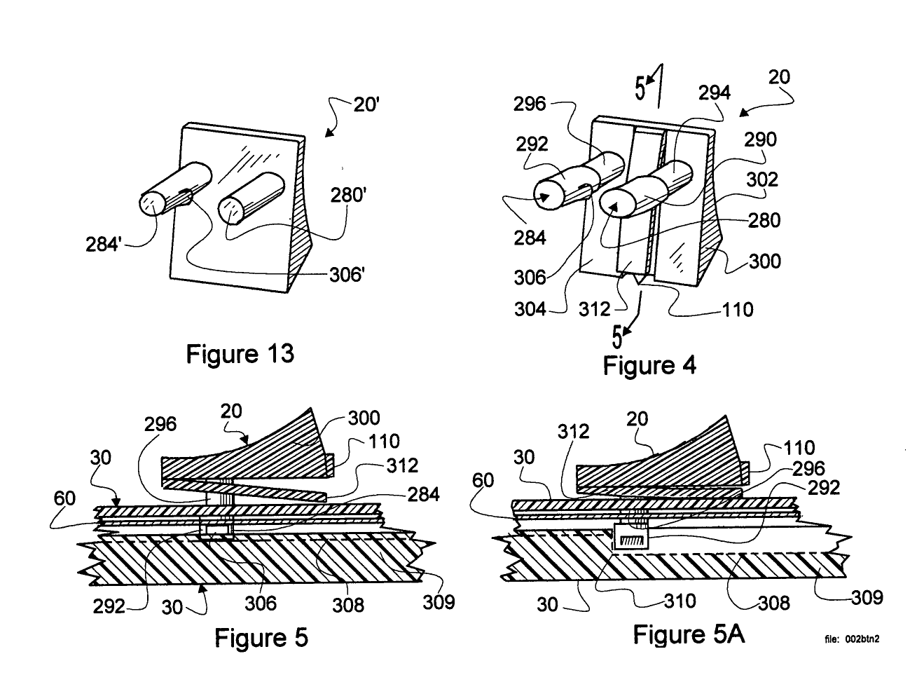

To facilitate appropriate and controllably restricted vertical displacement of actuator 20, stud 284 is disposed to slide in a groove (indicated by dashed line 308) disposed in a bottom part 309 of housing 30 (See FIG. 5). As long as groove 308 remains at the level seen in FIG. 5, section 292 remains in portion 286 of blade 60 (see FIGS. 3 and 3A). Section 290 is similarly retained in portion 282 of blade 60.

However, as seen in FIG. 5A, depth of groove 308 is abruptly changed at a site 310 which is disposed to cooperate with stop 299 (see FIG. 3A) to dispose thinner segment 296 of stud 284 in portion 292 thereby permitting stud 284 to be displaced along edge 212 of slot 210, as seen in FIGS. 3B-E. Stud 280 is likewise free for displacement along slot 200.

Clearly, as actuator 20 is distally displaced, each sharpened end 180 and 190 of blade 60 is seen to be arcuately displaced along hinge line 172. Note that full retraction of actuator 20 returns blade 60 into housing 30.

To make operation of actuator 20 more tactilely communicative, a leaf spring 312 is preferably added to base 304 of actuator 20, as seen in FIG. 4. Spring 312 resists depression of actuator 20 (and studs 280 and 284) into housing 30, there providing an impending sensation of scalpel state change as transition is made from the first to the second mode. Compressive action of actuator 20 is variously seen in FIGS. 5 and 5A. Note that compressing leaf spring 312 forces enlarged section 296 through slot 210, permitting transverse displacement of sharpened end 190 as seen in FIGS. 3B-E. Sharpened 180 is likewise displaced.

Actuator 20 and housing 30 may be injection molded from synthetic resinous materials. Actuator 20 may be molded from nylon or an acrylic while housing 30 may be molded from medical grade polypropylene. Parts of housing 30 which are securely affixed together to form a final housing which may be so affixed by adhesives, ultrasound, mechanical attachment or other methods which provide a secure bond. Such methods are well known in the medical device production art.

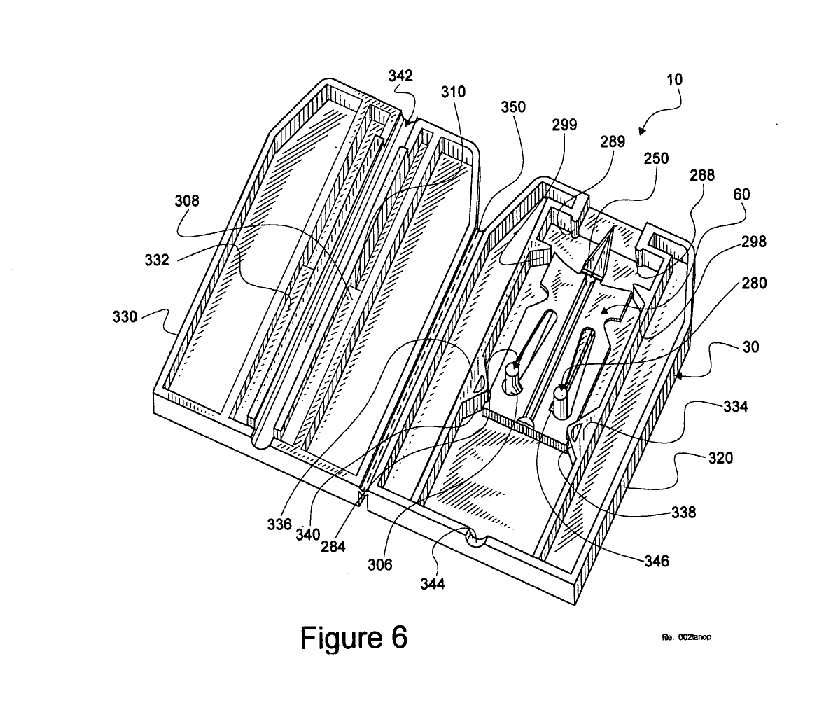

Reference is now made to FIGS. 6 and 8-10 wherein an open housing 30 is seen. As seen in FIG. 6, housing 30 may be fabricated as a unit mold comprised of a top part 320 and a bottom part 330. Note groove 308 in part 330 which was previously disclosed in FIGS. 5 and SA. Note that a similar groove 332 is disposed in part 330 as a guide for stud 284. Note also a pair of stops 334 and 336 which interface with a pair of arcuate indents 338 and 340 to provide a safety latch prior use.

Note an elongated semicircular groove 342 which forms a pathway for a guide-wire in part 330. Note also that a similar pathway is formed in part 320 by a proximally disposed notch 344 and channel 250 (see also FIG. 7B). Note also, that so assembled with blade 60 disposed to be held in place by latches 346 and 306 of studs 280 and 284, respectively, of actuator 20. Scalpel 10 may be delivered to a user in an open state, such as the state seen in FIG. 6. In such a case, parts 320 and 330 and joined by an elongated living hinge 350 and are designed to permanently snap closed after a guide-wire (generally referenced herein by the number 100) has been disposed therein (see FIG. 8). Such snap closures are well known in plastic molded parts design.

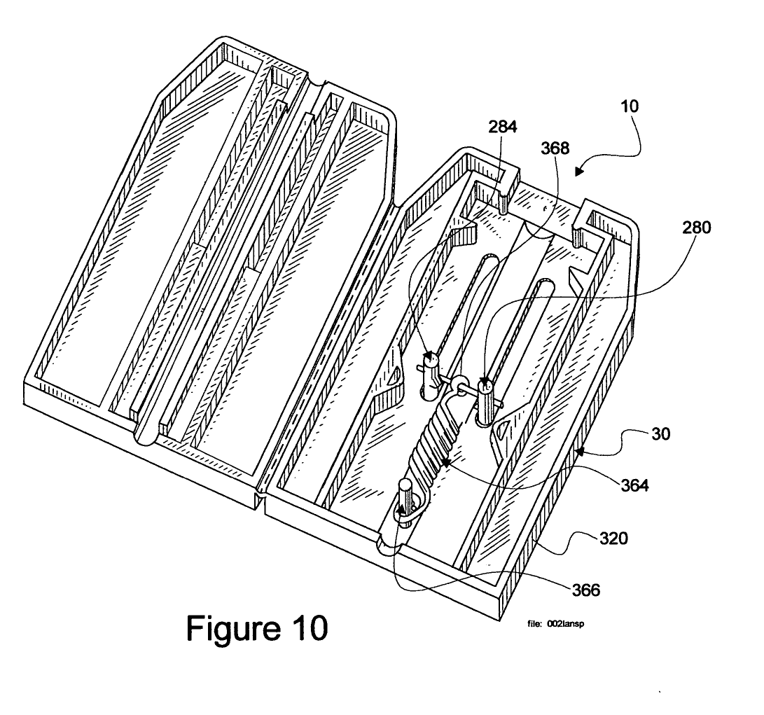

It may be preferable for housing 30 to be closed and sealed before delivery to a user. In such a case, a threadable pathway, such as one formed by a hollow straw 362 (see FIG. 9A) may be permanently affixed into groove 342, notch 344 and channel 250. Straw 362 is shown in dashed lines in FIG. 9.

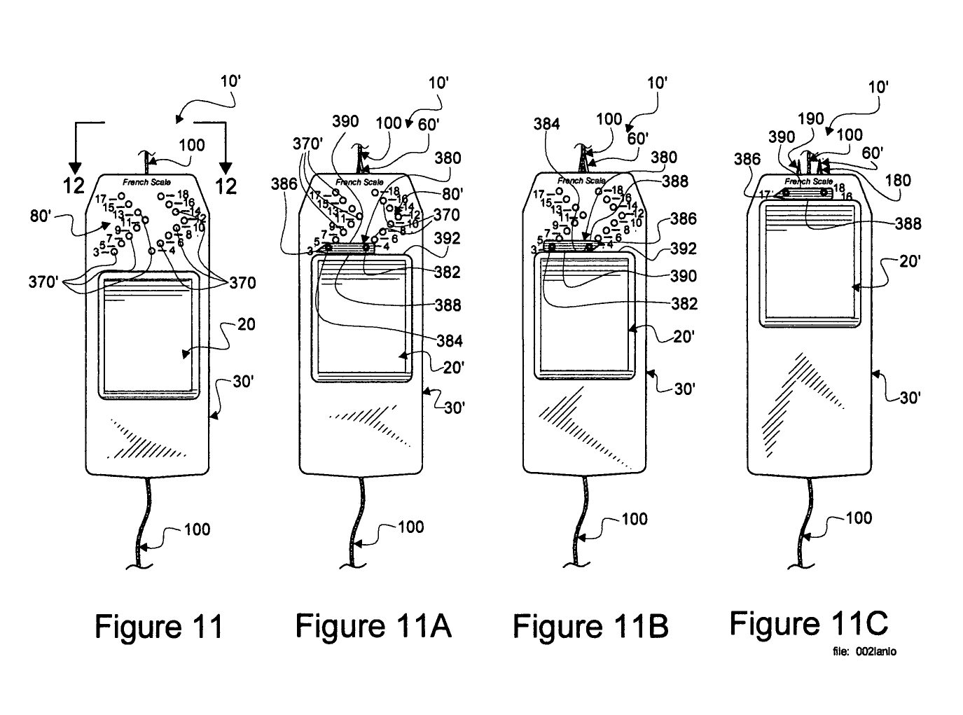

To provide an additional element of safety for scalpel 10, a spring 364 or other retracting element may be affixed between a post 366, seen projecting from an inner portion of top part 320, and therefrom to a coupling 368 in communication with studs 280 and 284 of actuator 20, as seen in FIG. 10. Note that displacement of actuator 20 relative to housing 30 extends spring 364, stores energy therein for forcibly returning actuator 20 to an original state as a user removes actuating force from scalpel 10. Of course, returning actuator 20 also returns blade 60 into housing 30. (Blade 60 is not shown in FIG. 10 to permit better visualization of disposition of spring 364.)

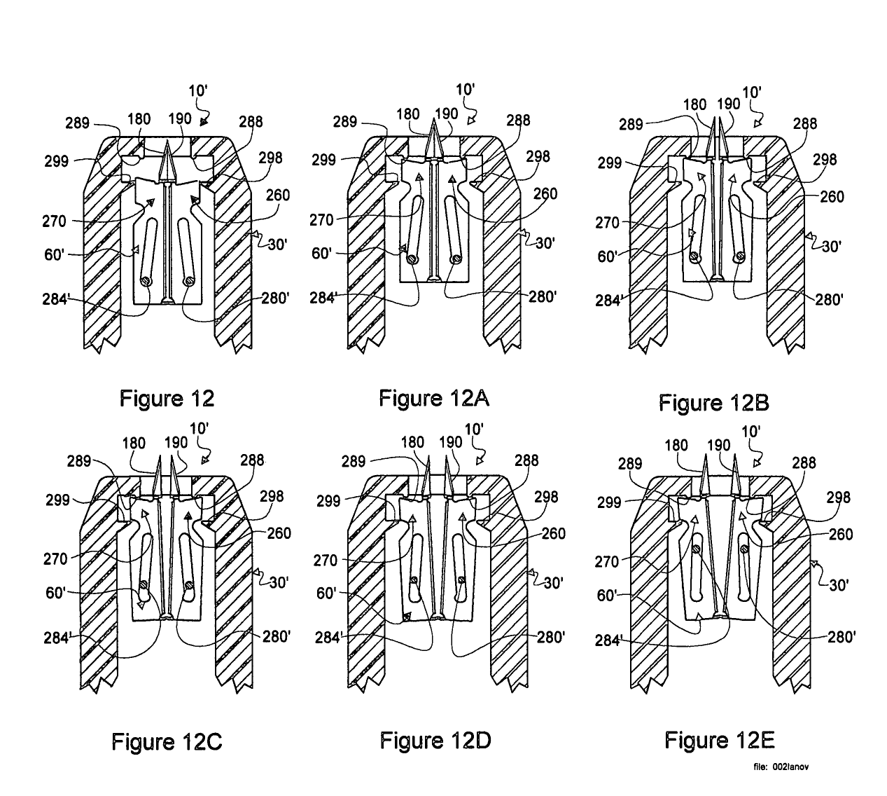

Reference is now made to FIGS. 11, 11A-11C, 12, 12A-12E, 13 and 14A. As disclosed supra, scalpels 10 and 10A are two mode devices (i.e. operate in two steps, requiring a user to perform a step of compressing actuator 20 against housing 30 (see FIGS. 5 and 5A) to enter the second mode. In the second mode, incision width is visually determined by displacing a pointer 110 relative to a set of indicia 80 disposed on a visible surface on top side 90 of housing 30. However, a scalpel may be made according to the invention in which the two modes operate in tandem without tactile feedback to a user. In such a case, it is considered prudent to provide a secure, physical stop which precludes over-widening an incision rather than relying on visual feedback.

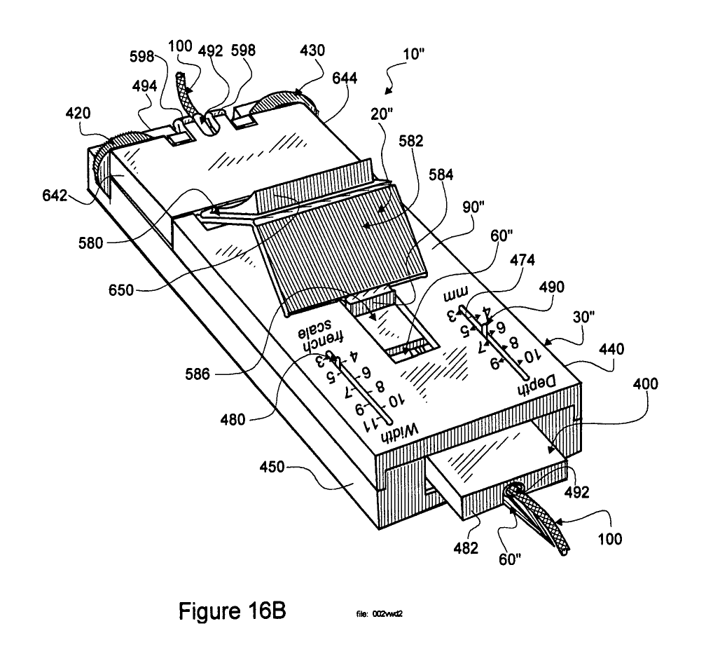

As seen in FIG. 11, scalpel 10′ appears similar to scalpel 10, but operates in a tandem fashion and has a physical stop which limits width of a resulting incision. A blade 60′ for scalpel 10′ is seen in FIG. 14A. Note that blade 60′ is similar in all ways to blade 60 (see FIG. 7B), except for differences between slots 200 and 210 and 200′ and 210′, respectively. Note that enlargement of slots 200′ and 210′ at respective proximal ends 204′ and 214′ are smaller in increased size than the corresponding sizes of proximal ends 204 and 214 of blade 60.

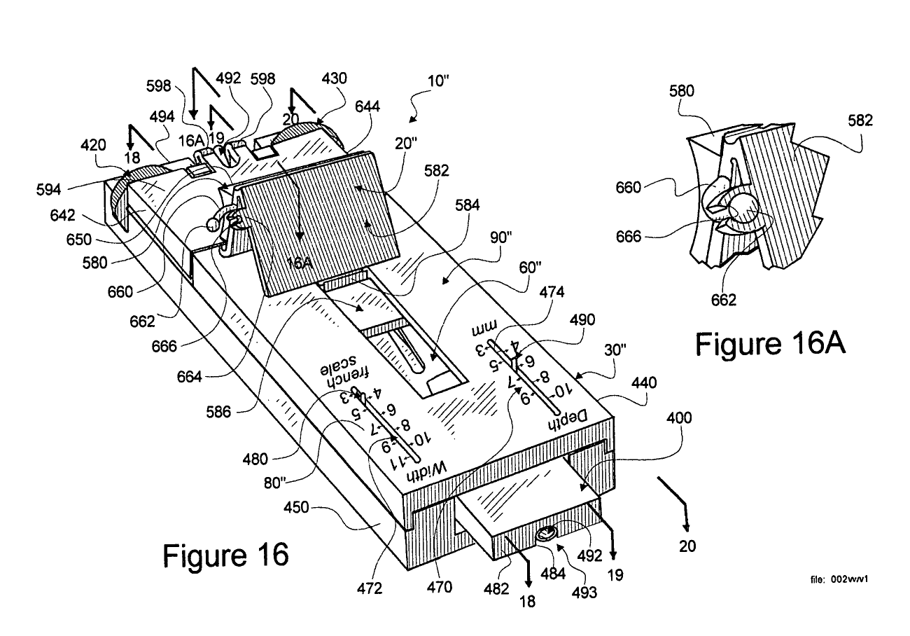

As is seen in FIG. 13, an actuator 20′ has studs 280′ and 284′ which are void of an enlarged segment, such as segments 290 and 292 of actuator 20. However, each stud 280′ and 284′ does have a latch, which corresponds to latch 306′ seen on stud 284′. Latches on studs 280′ and 284′ perform the same function as previously disclosed for latches 306 and 346. Needed space for insertion of latches through blade 60′ is the reason for any enlargement of proximal ends 204′ and 214′.

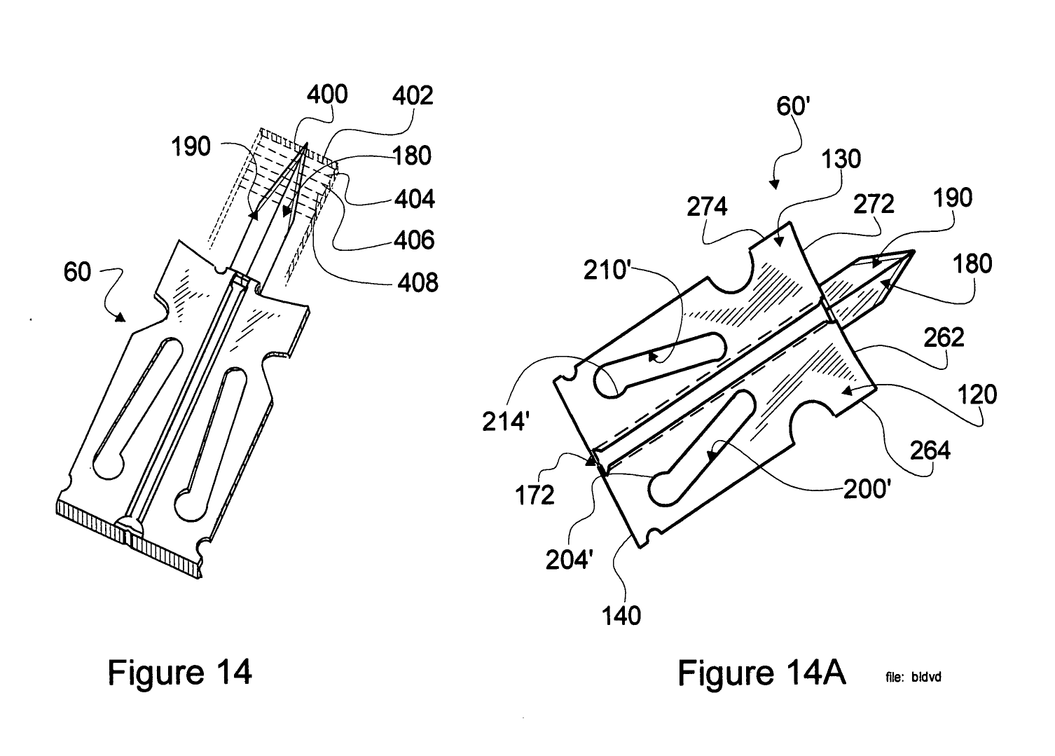

Note, in FIGS. 12 and 12A-12E, there is no physical impediment between studs 280′ and 284′. Sideways displacement of wings 260 and 270 is strictly limited by side stops 298 and 299, respectively (see FIG. 12), until wings 260 and 270 are retarded by stops 288 and 289, respectively (see FIGS. 12A-E). Note that width of separation of wing 260 and 270 and resulting separation of attached sharpened ends 180 and 190 of blade 601 is a linear function of distance of distal travel of studs 280′ and 284′ (as seen in FIGS. 12B-12E.

Scalpel 10′ provides an example of apparatus which utilizes a physical stop to limit width of an incision (see in FIGS. 11 and 11A-11B). A housing 30′ is seen in FIG. 11 to comprise a set of indicia 80′ with as set of corresponding pairs of holes, generally numbered 370 and 370′, each pair being disposed along a line orthogonal to the common plane, as defined supra. Note that one pair of holes corresponds to one pair of odd number and even numbers. The numbers as illustrated are french scale related values.

An adjustable and removable stop 380 is seen in FIG. 11A. Stop 380 is shown as a transparent part so pair of pegs 382 and 384 sized and positioned to fit into associated pairs of holes 370 and 370′ may be visualized. Stop 380 also comprises a pointer 386 and a pair of elongated sides 388 and 390. Sides 388 and 390 are asymmetrically disposed relative to pegs 382 and 384 such that when stop is disposed as seen in FIG. 11A side 388 abuts a distal end 392 of actuator 20′ to limit blade separation to a 3 french scale width. When stop 380 is rotated 180° and inserted into the same holes, side 390 abuts distal end 392 to limit blade separation to a 4 french scale width, as seen in FIG. 11B. Stop 380 may be variously disposed in each pair of holes 370 and 370′ to predetermine a desired maximum incision width, such as french scale 17 width seen in FIG. 11C.

Adjustable Width, Adjustable Depth Scalpel

Scalpels 10, 10A and 10′, disclosed supra, provide for making an incision of selectable width, but a constant, predetermined depth. Such scalpels are useful in well defined modes of application; however, it is highly desirable to provide a scalpel which is useful in a wide variety of catheter and other incision applications. Such applications may require a selection of both width and depth of incision settings.

As may be noted in FIG. 14, length of sharpened ends 180 and 190 may be extended to permit a longer travel of ends 180 and 190. Such travel permits use of a variably positioned nose barrier (nose 400) to limit an incision depth. Note that nose 400 may be variously disposed relative to extension of blade 60 travel as seen by dashed lines, variously numbered as 402, 404, 406 and 408. As charted, line 402 may limit depth at 3 millimeters. Lines 404, 408 and 410 may limit depth at 6, 9 and 12 millimeters, respectively.

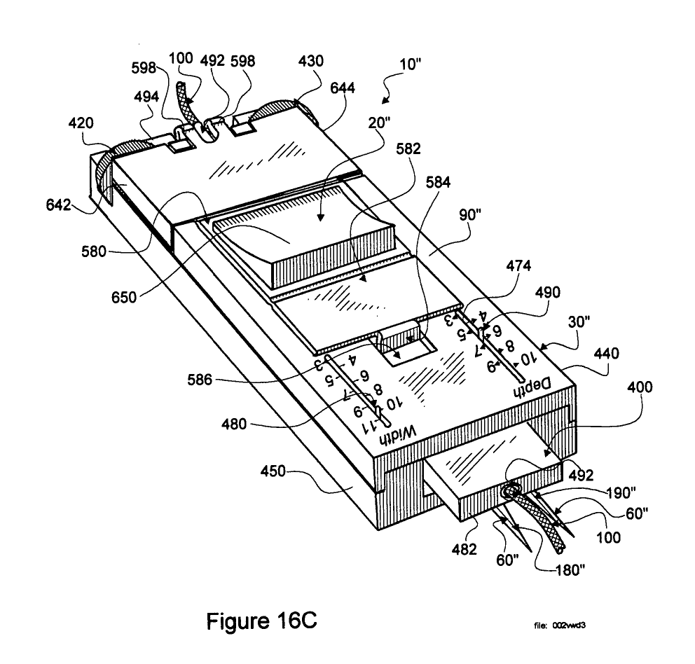

However, extending length of exposure of a scalpel blade from a housing requires extended effective displacement of an actuator relative to a housing. Such a displacement must be added to a length of displacement required to widen an incision. If all such blade related actuator displacement is linear, total effective actuator displacement may approach or go beyond an inch as may be seen by example of a scalpel 10B in FIG. 15 (compare with maximum displacement of actuator 20 in FIG. 1). Such an actuator displacement requirement may be physically difficult to manipulate in a single-hand operated device. For this reason, it deemed expedient to employ an actuator having a mechanical advantage for a variable width, variable depth scalpel. Note, also for reference hereafter, that scalpel 10B in FIG. 15 has an orifice 70B which provides an over-the-blade passage for a guide-wire.

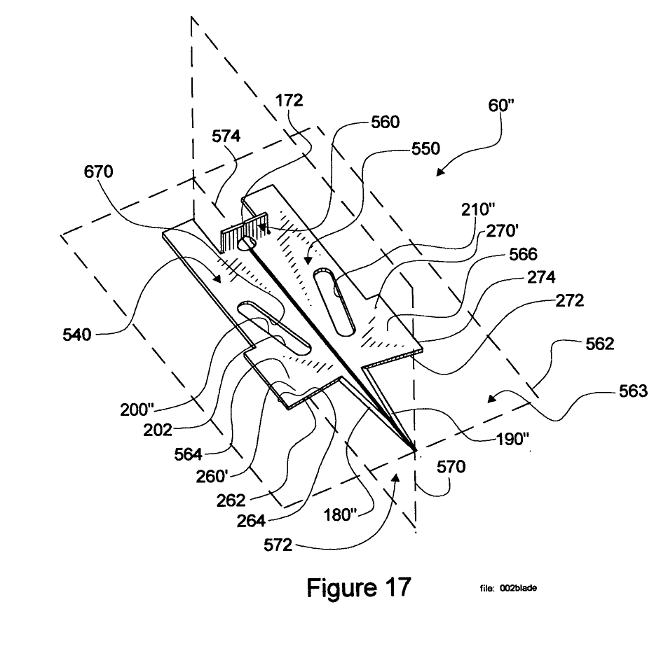

Reference is now made to FIGS. 16-22. In FIG. 16, an embodiment of a scalpel 10″, made according to the instant invention for producing incisions of both selected width and depth, is seen. Scalpel 10″ comprises an actuator 20″, a housing 30″, a thumbscrew apparatus 420 whereby incision width is set and restricted, another thumbscrew apparatus 430 whereby incision depth is set and restricted and an incision site interfacing nose 400.

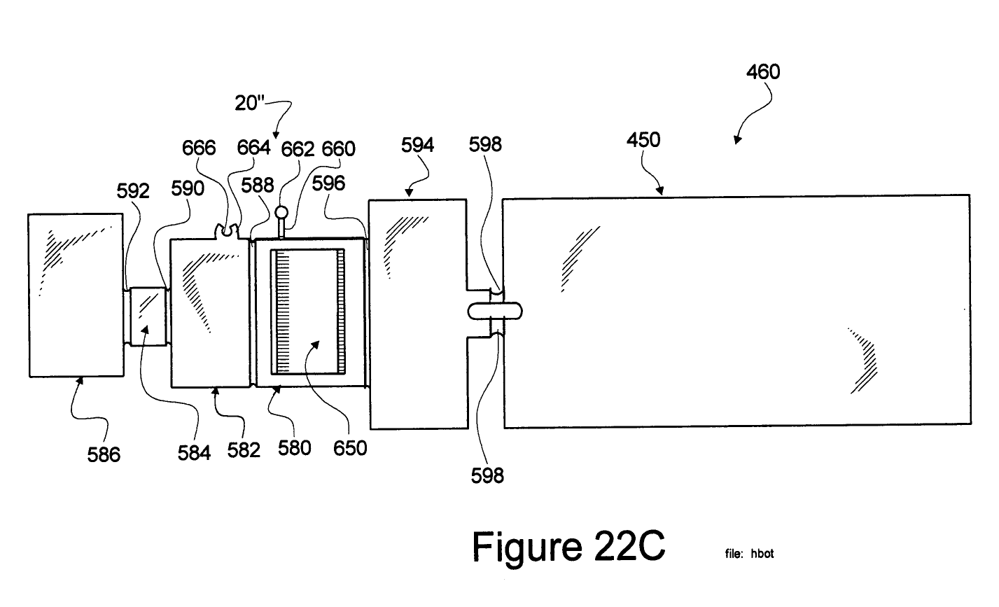

Housing 30″ comprises a distal top piece 440 and a bottom piece 450. For efficiency of manufacture, it is notable that bottom piece 450 and actuator 30″ may be molded as a unitary part 460 (see FIGS. 22 and 22A-22C). Distal top piece 440, unitary part 460 and nose 400 may all be made by injection molding. Synthetic resinous material such as polypropylene or other plastic from which living hinges may be created may be used.

Disposed upon a top side 90″ of housing 30″ are two sets of indicia, a first set, indicia 80″, and a second set, indicia 470. Associated with each set of indicia 80″ and 470 on top side 90″ is a respective elongated groove 472 and 474. Each groove 472 and 474 provides a track for respective portions of thumbscrew apparatus 420 and 430 disposed there below. An indicator pin 480, which is a part of apparatus 420, is seen protruding from groove 472. As an example provided by FIG. 16, pin 480 is disposed at indicia 80″ site “4”, indicating an incision width limit of a french scale 4. An indicator pin 490, which is a part of apparatus 430, is seen protruding from groove 474. Pin 490 is disposed at indicia 470 site “6”, indicating, by example, an incision depth limit of a 6 millimeters.

At its distal face 482, nose 400 comprises an opening 484 wherein a hollow straw 492 is disposed to provide a pathway 493 for a guide-wire 100. As may be seen on a proximal end 494 of housing 30″, straw 492 preferably extends distally to provide a pathway through scalpel 10″ for facile threading of a guide-wire 100.

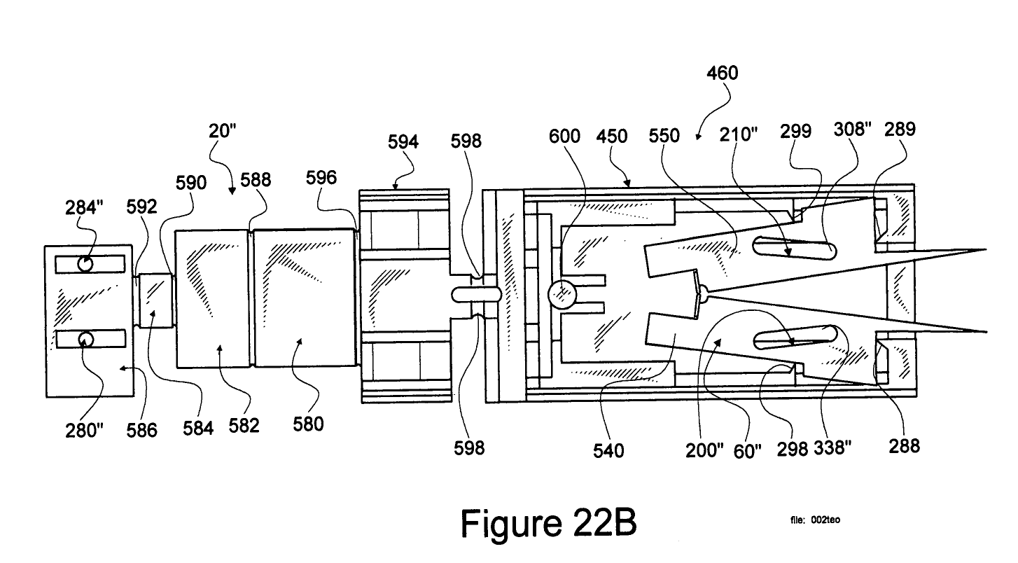

As seen in FIGS. 21 and 21A, nose 400 may be injection molded as a single, unitary part, except for straw 492. Straw 492 may be affixed to nose part 400 by adhesives, mechanical friction or in another manner which assures retention of straw 492 within housing 30″. While it may be possible to mold straw 492 as an integral part of nose part 400, angulation of straw 492 away from a plane of the top side 495 (see FIG. 21) of nose part 400 may make such molding inadvisable. Such angulation of straw 492 provides for a directed intersection between a guide-wire 100 and tips of sharpened ends 180 and 190 of an associated scalpel blade.

An elongated slit 496 may be seen disposed along top side 495 of nose 400 where through pin 480 (seen in FIG. 16) extends as a width delimiter and indicator. A hole 498 on a side opposite slit 496 provides a access for a protruding pin 490 (also see FIG. 16).

In FIG. 21A, where nose part 400 is rotated for a view of bottom side 499, nose part 400 is seen to comprise two elongated channels 500 and 502. Channel 500 comprises a pair of substantially parallel side rails 504 and 506 which are closed by a head rail 508. A pair of latching rails, each numbered 510 and 512, extend inward from rails 504 and 506, respectively, to form, in cooperation with rails 504, 506 and 508, an open compartment 513. At a proximal end, channel 500 is open for disposition therein of thumbscrew apparatus 430, as seen in FIGS. 16 and 21A.

Apparatus 430 comprises a thumbscrew 522 which comprises an internal thread into which an elongated threaded rod 524 is disposed. Securely affixed to a distal end 526 of rod 524 is a rectangularly shaped plate 528. Plate 528 is sized and shaped to snugly fit into compartment 513 with pin 490 extending through hole 498. Note that, when thumbscrew 522 is anchored and turned, nose part is displaced by proximal and distal displacement of rod 524 and plate 528.

Channel 502 comprises a pair of elongated side rails, numbered 530 and 532, providing an unrestricted channel for displacement of apparatus 420. Apparatus 420 is like apparatus 430, except for numbering of pin 480 to allow differentiation from pin 90 of apparatus 430. Apparatus 420 is not seen in FIG. 21A. However, it should be noted that pin 480 is free to slid along groove 496 without affecting displacement of nose part 400.

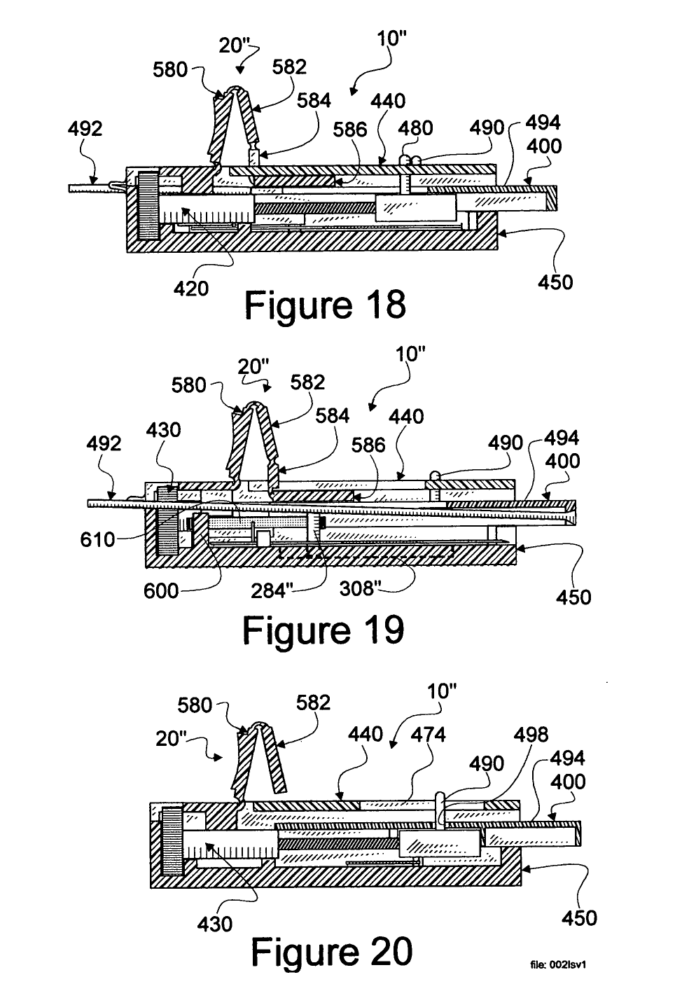

Reference is now made to FIG. 17 wherein a scalpel blade 60″, also made according to the invention, is seen. Note that blade 60″ may be considered to be of two mirror image related parts, 540 and 550, interconnected by a hinge part 560. Similar to blade 60′ (see FIG. 14A), blade 60″ comprises a pair of guide slots 200″ and 210″. However, note that there is no end disposed enlargement of slots 200″ and 210″. Blade 60″ also comprises a pair of sharpened ends, 180″ and 190″, which are substantially more elongated than ends 180 and 190. Hinge part 560 is shorter than respective hinge part 140 (see FIGS. 7, 7A, 7B and 14A), but is thinned about a hinge line 172 to provide for facile articulation of respective parts 540 and 550.

Also, in FIG. 17 two planes are outlined by dashed lines. Dashed line 562 defines a plane 563 congruent with top surfaces 564 and 566 of parts 540 and 550, respectively. Plane 563 is like the common plane defined supra. Dashed line 570 defines a plane 572 orthogonal to plane 563, creating an intersecting line 574 which divides mirror imaged parts 540 and 550.

Like blades 60 and 60′, blade 60″ is preferably made from medical grade stainless steel having sufficient thickness that sharpened ends 180″ and 190″ do not bend when performing an incision. Also, slots 200″ and 210″ accept and act against studs disposed therein to first displace blade 60″ distally and then laterally as an incision is made.

Also, similar to blades 60 and 60′, blade 60″ comprises leading edges 262 and 272 for limiting extension of blade 60″ outwardly distally from housing 30″ and for compensating for depth variations as ends 180″ and 190″ are articulated, thereby maintaining a substantially constant incision depth. Likewise, edges 264 and 274 act against associated side stops in housing 30″ to keep parts 540 and 550 parallel to orthogonal plane 572 throughout initial or the first distal displacement mode of operation. At proximal ends of edges 264 and 274, parts 540 and 550, are respectively indented to provide relief against the side stops so that parts 540 and 550 may articulated in the second mode of operation.

Reference is once again made to FIG. 16 wherein actuator 20″ may be seen to comprise a plurality of hinged parts. Actuator 20″ comprises a digitary actuator plate 580, a second plate 582, a feed-through plate 584 and a stud plate 586.

Better seen in FIG. 22, plates 580, 582, 584 and 586 are respectively interconnected by living hinges 588, 590 and 592. Such living hinges (and other living hinges disclosed herein, with the exception of steel hinge at line 172 of blades 60, 60′ and 60″, may be created as a product of injection molding. Such processes are well known in the injection molding art. Also plates 582 and 584 may be the same plate, eliminating hinge 590.

Actuator plate 580 is affixed to a proximal housing top piece 594 via a living hinge 596. Housing top piece 594 is affixed to bottom piece 450 of house 30″ via a double hinge (generally numbered 598). Blade 60″ is also seen disposed in bottom piece 450 in a most proximal position in FIG. 22. Note side stops 298 and 299. Not also distally disposed stops 288 and 280. These stops are similar in function to the same numbered stops in seen in FIGS. 3 and 12.

Stud plate 586 is seen to comprise a pair of studs 280″ and 284″. Actuator 20″ is designed to be folded and to insert studs 280″ and 284″ into slots 200″ and 210″ and there through into guide-slots 332″ and 308″, respectively. So disposed, distal displacement of stud plate 586 first displaces a closed blade 60″ to a most distal site as seen in FIG. 22A. Further displacement of stud plate 586 articulates parts 540 and 550 as seen in FIG. 22B. Of course, such blade 60″ displacement takes place within a fully assembled scalpel 10″.

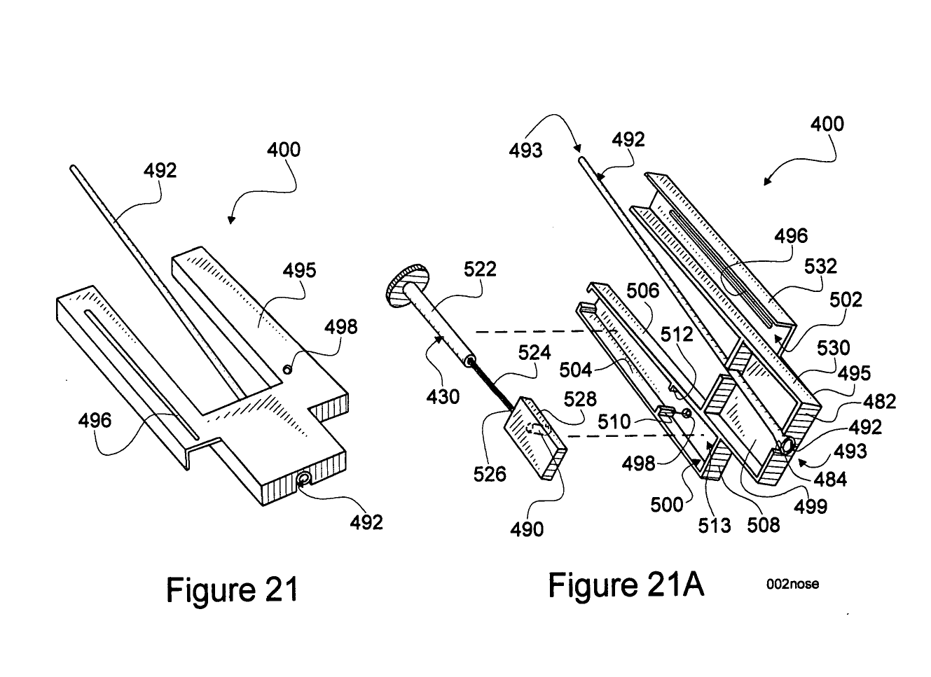

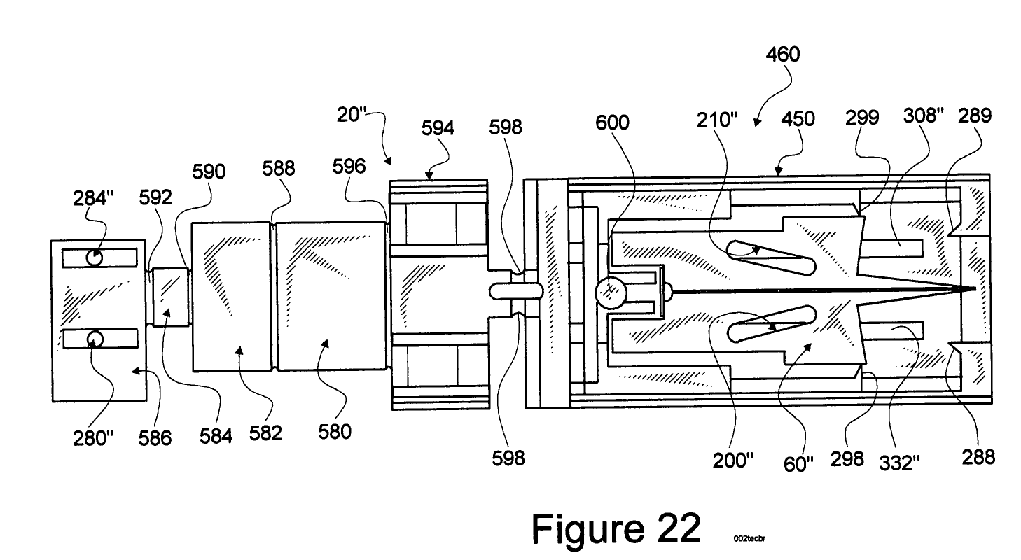

Assembly of scalpel 10″ is variously seen in FIGS. 16, 18-20, 21, 21A and 22. As seen in FIG. 22, blade 60″ is placed in a proximal site in bottom piece 450 of housing 30″. Thumbscrew apparatus 430 is affixed into compartment 513 of nose part 400 with pin 490 displaced through hole 498 as indicated in FIG. 21A. Thumbscrew apparatus 420 is likewise displaced into channel 502 with pin 480 displaced through groove 496 as seen in FIG. 18. So assembled, nose part 400 and thumbscrew apparatus 420 are displaced into bottom piece 450. Note that straw 492 is displaced through an open portion of hinge 598 as seen in FIGS. 16 and 19. Note also disposition of thumbscrew apparatus 430 in FIG. 20 along with associated pin 490 disposed in groove 474.

For optional automatic retraction of blade 60″, a post 600 (see FIG. 22) is molded into bottom piece 450. As disclosed supra, a retraction mechanism may be a spring. However, in this embodiment, a high grade elastic band which has a long life and characteristics that withstand sterilization is preferred. Such an elastic band 610 is seen disposed about post 600 and a stud 284″ in FIG. 19. As studs 280″ and 284″ are displaced distally, energy is stored in band 610 and recovered to retract blade 60″ when force against actuator 20″ is released.

Proximal housing top piece 594 is articulated about hinge 598 to form a portion of the top of housing 30″ as seen in FIG. 16. Plates 586, 584, 582 and 580 are articulated as seen in FIG. 18 with band 610 being wrapped about studs 280″ and 284″ which are thereafter displaced through slots 200″ and 210″ and further displaced into grooves 332″ and 308″, respectively (see FIG. 22). Note, also, that pin 480 is disposed to contact and impede continued distal displacement of stud plate 586, thereby predetermining and limiting incision width.

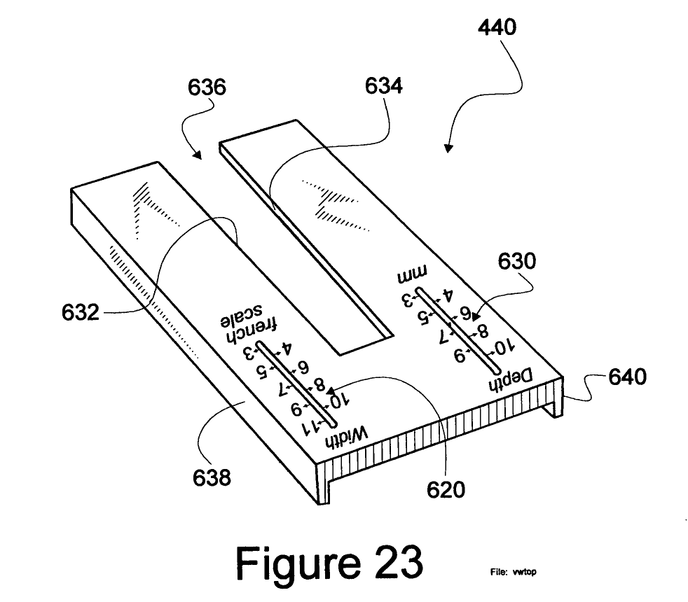

Distal top piece 440, seen separately in FIG. 23, comprises slots previously disclosed. Distal top piece 440 is a “U” shaped part comprising width related indicia 620 and depth related indicia 630. Proximal extensions 632 and 634 of distal top piece 440 define a medial opening slot 636 which is sufficiently wide to permit feed through plate 584 to be displaced there along, but too narrow for similar displacement of plates 582 and 586. Each extension 632 and 634 comprises an inferiorly disposed ledge 638 and 640, respectively, which are sized and shaped to interconnect with corresponding parts of bottom piece 450 to provide a unitary housing 30″ assembly. In similar fashion, proximal top piece 594 has similar inferiorly disposed ledges 642 and 644 (see FIG. 16) which interconnect with bottom piece 450.

As is seen in FIGS. 16, 16B, 16C and 22C, scalpel 10″ preferably comprises an articulating digitary interface 650 disposed upon an accessible side of plate 580. Curvature of interface 650 should permit either thumb or finger actuation thereby allowing under guide-wire 100 and over guide-wire use of scalpel 10″.

Advantage of a folded actuator 20″ over a linear actuator, such as actuator 20, is easily seen when considering increased travel distance of stud plate 586 compared with travel distance required for actuator 20. Actuator 20 must only travel a limited distance (e.g. 3 millimeters) in the first mode and, in the maximum, on the order of a centimeter in the second mode. However, for a variable incision width, variable depth scalpel made according to the invention, stud displacement may exceed two centimeters. For some users, two centimeters digitary displacement may not be acceptable. Thus, the mechanical advantage offered by a folded actuator, such as actuator 20″ permits a relatively long stud displacement with a much shorter digitary displacement. Note in FIGS. 16, 16B and 16C that actuator plate 580 only rotates from a near vertical position (FIG. 16) to a horizontal position (FIG. 16B) to fully extend and separate sharpened ends 180″ and 190″ of blade 60″.

Stability of scalpel 10″, prior to use, is very important. Blade 60″ must be retained within housing 30″ until a user is ready to make an incision. For this purpose, it is preferred to provide a releasible latch which is unlatched just prior to use. Such a latch may be variously seen in FIGS. 16, 16A and 22C. As seen in FIG. 22C, an outwardly extending rod 660 which is flexible as it is made from the same synthetic resinous material used for living hinges. Rod 660 is terminated by a bulbous globe 662. At complementary site on plate 582, a protrusion 664 comprises an open arcuate notch 666 sized to receive and latch rod 660 therein. As seen in FIG. 16A, rod 660 is disposed in notch 666 to securely latch plate 580 to 582. In FIG. 16 rod 660 is freed from notch 666 so actuator 20″ may be used to propel and articulate scalpel 60″.

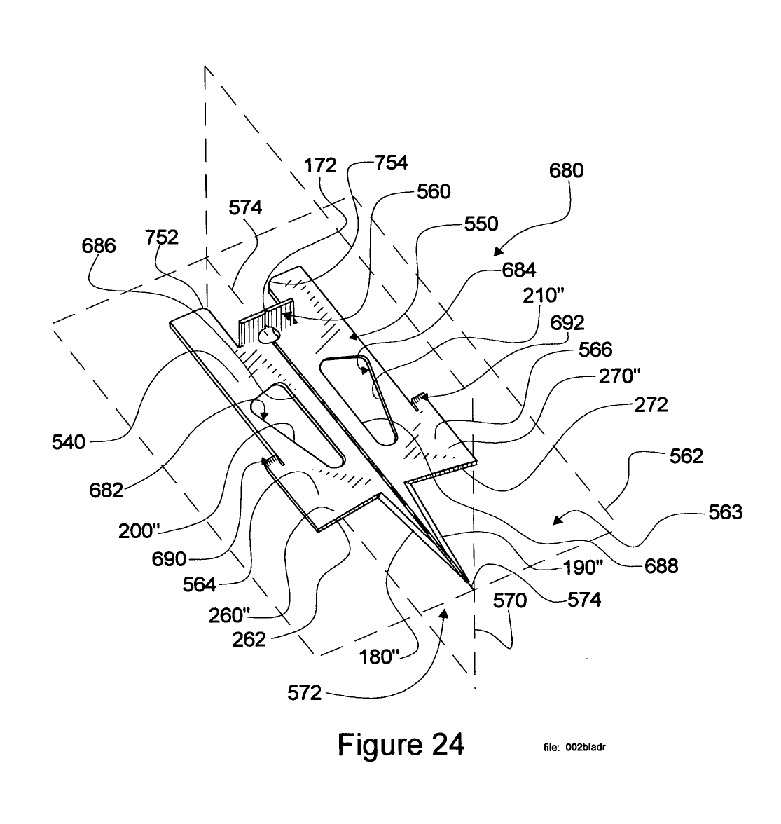

Reference is now made to FIGS. 24-26B wherein yet another embodiment of the invention is seen. This particular embodiment addresses potential problems of a closing scalpel blade seizing a guide wire while retracting. This scalpel is referenced as scalpel 10A. Note that previously disclosed blades 60, 60′ and 60″ have guide slots (i.e. slots 200, 200′ and 200″ and 210, 210′ and 210″, respectively) which are seen to have juxtaposed, parallel elongated edges. As an example, in FIG. 17, slot 200 is bounded outwardly by edge 202 and inwardly or more medially by edge 670. As disclosed supra, when actuator 20″ is distally displaced, stud 284″ acts against edge 202 to displace sharpened end 180″ outwardly. In consequence, when actuator 20″ is displaced proximally, stud 284″ acts against edge 670 to displace end 180″ medially. Thus, if sharpened ends 180″ and 190″ are medially displaced before blade 60″ is proximally displaced away from a guide wire 100, ends 180″ and 190″ may close together and seize guide wire 100.

A blade 680 is seen in FIG. 24. Blade 680 is substantially the same as blade 60″ with two exceptions. The first exception is seen to be a change of form of slots. Changed slots are numbered 682 and 684 in blade 680. Rather than elongated slots having elongated parallel edges as seen of slots 200″ and 210″ in FIG. 17, slots 682 and 684 have medial edges 686 and 688, respectively, which are disposed to be relieved from contact with a retracting stud. Thus, when a stud disposed therein, is retracted, no force upon either edge 686 or 688, by a retracting stud, acts to close sharpened ends 180″ and 190″.

The second exception is seen to be wing structure of blade 680. Note in FIG. 17 that wings 260 and 270 of blade 60″ are substantially planar in construction. Rather, wings 260″ and 270″ depart from planar construction at proximal edges 690 and 692, respectively. As may be perceived from shading on each proximal edge 690 and 692, wings 260″ and 270″ are respectively bent upwards thereat.

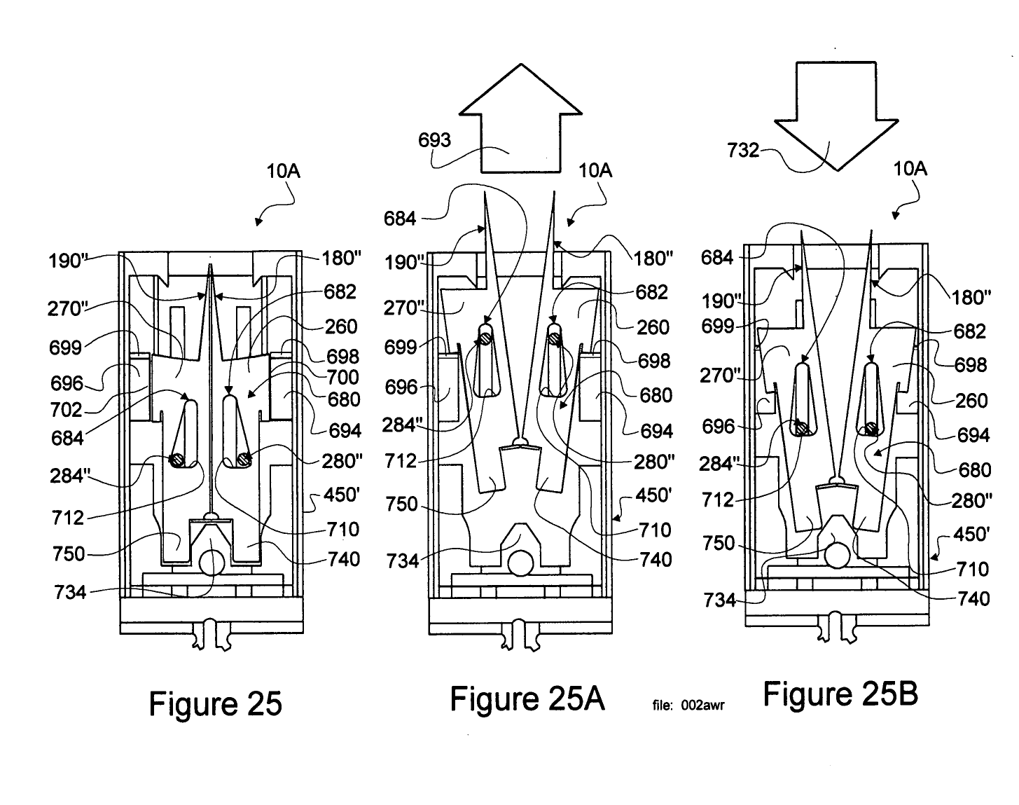

Referring to FIG. 25, blade 680 is disposed in a bottom piece 450′ of a scalpel 10A. Bottom piece 450′ is like bottom piece 450, disclosed supra, except for two exceptions. As a first exception, bottom piece 450′ has a pair of guide blocks 694 and 696. Each guide block 694 and 696 has a respective rounded distal edge 698 and 699. Each guide block 694 and 696 also has a respective medial edge 700 and 702 which acts against respective wings 260″ and 270″ to constrain blade 680 in a closed state while there is contact therewith. (See FIG. 25.)

Once blade 680 is distally displaced, in direction of arrow 693, such that wings 260″ and 270″ are free of guide blocks 694 and 696, studs 280″ and 284″ force sharpened ends 180″ and 190″ apart, as seen in FIG. 25A. However, when studs 280″ and 284″ are proximally displaced, no contact is made against perimeter portions of slots 682 and 684, respectively until first contact is made with each proximal edge 710 and 712 of respective slots 682 and 694. (See FIG. 25B for contact sites.)

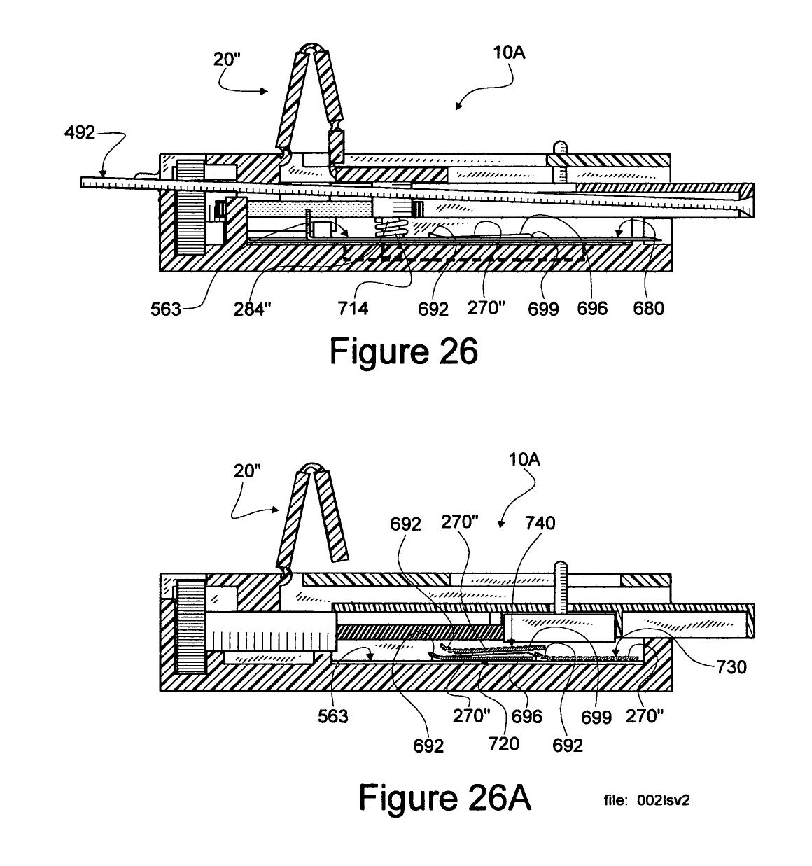

Referring to FIG. 26, wing 270″ of blade 680 is seen to be nested against stop 696 and urged into retention thereat by a spring 714 disposed about stud 284″. (See also FIG. 25.) Thus, wing 270″ is substantially disposed in common plane 563, with the exception of upward bending of edge 692. A cross section of wing 270″ is seen so disposed at site 720 in FIG. 26A. Similarly, wing 270″ remains in plane 563 when wing 270″ is distally displaced to site 730, also seen in FIG. 26A.

However, when blade 680 (and wing 270″) is proximally displaced (in direction of arrow 732 of FIG. 25B), curvature of edge 692 acts against rounded distal edge 699 to force wing 270″ away from plane 563. Wing 270″ (and wing 260″) therefore ride above and over respective stops 694 and 696, respectively, keeping blade 680 open, as seen in FIG. 25B, and thereby are not displaced medially until blade is nearly fully retracted.

The second exception of bottom piece 450′ is a proximal “V” shaped guide block 734, which act against proximal skirts 740 and 750 of to close sharpened ends 180″ and 190″ together as blade 680 is proximally displaced to an original or starting state as seen in FIG. 25. In this manner, blade 680 is displaced as two modes of operation in making an incision, being thrust forward in a closed state until reaching a predetermined depth of incision and then driving sharpened ends apart to widen the incision to a predetermined width. Also blade 680 operates in basically two mode during retraction, being proximally returned to the housing in an open state and being closed and nested at a final stage of retraction. Note that relative medially disposed corners 752 and 754 of skirts 740 and 750 (see FIG. 24) are rounded for a less intrusive interface when in contact with block 734.

In some split blade scalpels, it may be desired to form an incision which is wider at the widest extremity of the incision than closer to orthogonal plane 572, see FIG. 24. To provide for such a form of incision, edges 262 and 272 are more designed to form a more acute angle with orthogonal plane 572 than for a square or incision of constant depth.

The invention may be embodied in other specific forms without departing from the spirit or essential characteristics thereof. The present embodiments are therefore to be considered in all respects as illustrative and not restrictive, the scope of the invention being indicated by the appended claims rather than by the foregoing description, and all changes which come within the meaning and range of equivalency of the claims are therefore intended to be embraced therein.

1. A planar, split scalpel blade element for producing an incision having a predetermined width and a predetermined, substantially constant, predetermined depth, said blade element comprising:
two juxtaposed elongated components aligned in a first plane with each component split away from the other component along a medial second plane which is orthogonal to the first plane, when medially aligned, said components jointly forming a distally disposed piercing end and comprising a common hinged joint aligned with the second plane at a proximal end;
a first component of the two components comprising a first knife blade at the piercing end, which is sharpened to be effective as a piercing and transverse cutting tool, and an inwardly disposed elongated first edge which is angularly displaced relative to the second plane to thereby provide a guide by which the piercing end of the component is skewed away from the second plane as a part of an actuator is displaced there along in a direction substantially parallel to the second plane toward the piercing end;
a second component of the two components comprising a second knife blade at the piercing end, which is sharpened to be effective as a piercing and transverse cutting tool, and an inwardly disposed elongated second edge which is angularly displaced relative to the second plane to thereby provide a guide by which the piercing end of the component is skewed away from the second plane as a part of an actuator is displaced there along in a direction substantially parallel to the second plane toward the piercing end;
the first and second component each comprising a third and fourth edge, respectively, each third and fourth edge being respectively disposed transversely outward from said first edge and second edge, each third and fourth edge comprising an elongated substantially straight length which is substantially parallel to the second plane when the components are juxtaposed and aligned with the second plane to thereby provide a pair of guides which interact with associated side stops which are fixed relative to blade movement and which obstruct sideways displacement of the first and second components thereby causing each actuator part and blade component to move distally as a unit without outward skewing of the first and second components until the blade element is distally displaced to the predetermined depth;
said first component further comprising a first wing defined by a first distal edge outwardly disposed between a proximal segment of said first knife blade and the distal end of the straight length of the third edge, said first wing further comprising a first arcuate, concave edge which is joined to a proximal end of the third edge, the first distal edge providing an interface against an associated distal stop which limits forward displacement of the first component whereat the first concave edge is disposed to provide relief from the associated side stop of the third edge to thereby permit outward skewing of the first component as the actuator part continues to be distally displaced and thereby skew the first knife blade outward, away from the second plane, to widen the incision to the portion of the predetermined width formed by the first component of the blade; and
said second component further comprising a second wing defined by a second distal edge outwardly disposed between a proximal segment of said second knife blade and the distal end of the straight length of the fourth edge, said second wing further comprising a second arcuate, concave edge which is joined to a proximal end of the fourth edge, the second distal edge providing an interface against an associated distal stop which limits forward displacement of the second component whereat the second concave edge is disposed to provide relief from the associated side stop of the fourth edge to thereby permit outward skewing of the second component as the actuator part continues to be distally displaced and thereby skew the second knife blade outwardly away from the first knife blade to widen the incision to the portion of the predetermined width formed by the second component of the blade.

2. A planar, split scalpel blade element according to claim 1 wherein the first transverse distal edge comprises a curvature which acts to maintain a constant depth of penetration of the proximal end of the scalpel blade as the first component is transversely displaced against the associated distal displacement limiting stop.

3. A planar, split scalpel blade element according to claim 1 wherein the second transverse distal edge comprises a curvature which acts to maintain a constant depth of penetration of the proximal end of the scalpel blade as the second component is transversely displaced against the associated distal displacement limiting stop.

4. A wire steered scalpel for enlarging catheter entry sites wherein a guide wire has been inserted preparatory to introducing a catheter, said scalpel comprising:
a housing comprising a pathway having an entry portal and an exit portal through which the guide wire is threadably disposed to facilitate steering the housing to the entry site, the entry portal being proximal to the entry site when the scalpel is used;
a split scalpel blade having a pair of sharpened points, said blade being within the housing and aligned with the pathway to lance, at the entry site, to be extended from the housing and thereby produce an incision having a predetermined width and depth; and
an actuator comprising a plurality of blade interfacing parts by which the scalpel blade is manually displaced relative to the housing and pathway to cause the blade to lance.

5. A wire steered scalpel according to claim 4 wherein the scalpel blade is medially split to comprise two scalpel components aligned to provide a distally disposed piercing end and a proximally disposed end whereat the two components comprise a common hinge connection.

6. A wire steered scalpel according to claim 5 wherein the housing comprises stops and the blade comprises guide edges which interface to the actuator parts and side stops to maintain the scalpel components aligned until the scalpel pierces the entry site to the predetermined depth.

7. A wire steered scalpel according to claim 4 wherein the housing comprises at least one stop for limiting travel of the scalpel blade components, thereby limiting depth of the lance and ending a first lancing motion of the scalpel.

8. A wire steered scalpel according to claim 4 wherein the guide edges of the blade comprise relief for the side stops when the scalpel blade reaches the at least one blade travel limiting stop, thereby permitting the components to be displaced. transversely relative to direction of displacement of the actuator, thereby widening the incision as a second motion of the scalpel blade.

9. A wire steered scalpel according to claim 4 wherein the blade comprises at least one distally disposed wing which comprises an edge which interfaces with the at least one stop, the interfacing edge having a curvature which substantially maintains a constant depth of incision as each blade component is transversely displaced.

10. A wire steered scalpel according to claim 4 wherein said housing in cooperation with said actuator define selectable stops whereby actuator travel is limited and, as a result, transverse displacement of the components is limited to selectively control and limit width of an incision.

11. A wire steered scalpel according to claim 4 wherein said housing comprises a top part and a bottom part.

12. A wire steered scalpel according to claim 11 wherein said top and bottom parts are molded as a single unit wherein said parts comprise a common living hinge which permits the parts to close together as a clam shell.

13. A wire steered scalpel according to claim 12 wherein said scalpel blade comprises a medially depressed channel into which a guide wire is displaced preparatory to closing the top and bottom parts together.

14. A wire steered scalpel according to claim 12 wherein said actuator parts comprise latches which act against the scalpel blade to secure the blade to a part of the housing while the housing is open.

15. A wire steered scalpel according to claim 4 wherein said housing comprises an adjustable distally disposed nose part comprising a distal face which is adjustable along a longitudinal medial line to selectably limit scalpel blade displacement distally beyond the distal face and thereby limit depth of penetration of the blade.

16. A wire steered scalpel according to claim 4 wherein said housing comprises an enclosed pathway for a guide wire threaded there through, said pathway having a distal opening and a proximal opening whereby the scalpel is displaced along the guide wire to an incision site.

17. A wire steered scalpel according to claim 16 wherein said housing comprises a pathway offset to provide a point of coincidence between the guide wire and the sharpened point of a fully extended scalpel blade.

18. A wire steered scalpel according to claim 4 wherein said actuator comprises a plurality of hingeably interconnected parts, including a manual interface component, which cooperate to provide a mechanical advantage whereby linear displacement of the scalpel blade is greater than manual displacement of the interface component.

19. A wire steered scalpel according to claim 18 wherein said actuator parts are molded as a single part comprising living hinges therewith molded.

20. A wire steered scalpel according to claim 18 wherein said actuator comprises a living hinge connection produced by intregrally molding at least one housing part with the actuator.

21. A wire steered scalpel according to claim 18 further comprising a spring affixed on a first end to the housing and to actuator on a second whereby the blade is automatically returned into the housing after use as a safety feature.

22. A method for making a carefully controlled incision at an entry site comprising the steps of:
(a) providing a guide-wire steered scalpel, said scalpel comprising:
a housing comprising a pathway having an entry portal and an exit portal through which a guide-wire is threadably disposed to facilitate steering the housing to the entry site, the entry portal adjoining the entry site when the scalpel is used;
a split scalpel blade, having two sharpened blades and associated points, which is disposed within the housing and aligned with the pathway to lance at the entry site, said blade being extended from the housing to thereby produce an incision having a predetermined width and depth; and
an actuator comprising at least one blade interfacing part by which the scalpel blade is manually displaced relative to the housing and pathway to cause the blade to lance;
(b) displacing a guide-wire which has been previously inserted into an entry site through the entry and exit portals, respectively;
(c) displacing the housing along the guide-wire to the entry site; and
(d) actuating the scalpel blade to pierce the entry site to a predetermined depth and to a predetermined width.

23. A method according to claim 22 wherein step d comprises two separate steps as follows:
(e) extending the scalpel blade from the housing to produce an incision to the predetermined depth; and
(f) a second mode whereby the scalpel blade is split apart to widen the incision to a predetermined width.

24. A method according to claim 23 wherein transition from the first mode to the second mode is continuous and subliminal to a user actuating the scalpel.

25. A method according to claim 22 comprising a further step of retracting the scalpel blade into the housing at the end of the actuating step.

26. A method according to claim 25 wherein the actuating step comprises a step which stores energy in an elastic memory device.

27. A method according to claim 26 comprising an additional step of automatically retracting the scalpel blade into the housing due to release of energy stored in the elastic memory device at the end of the actuating step.

28. A method according to claim 25 wherein the scalpel blade retracting step comprises the following steps:
(g) returning the two scalpel blades to an original juxtaposed state; and
(h) retracting the entire scalpel blade into the housing.

29. A method according to claim 28 wherein step h occurs following completion of step g.
